(19) United States
(12) Patent Application Publication
(10) Pub. No.: US 2018/0135120 A1
(43) Pub. Date: May 17, 2018

(54) COMPREHENSIVE METHODS FOR DETECTING GENOMIC VARIATIONS

(71) Applicant: The Jackson Laboratory, Bar Harbor, ME (US)

(72) Inventor: Yijun Ruan, Farmington, CT (US)

(73) Assignee: The Jackson Laboratory, Bar Harbor, ME (US)

(21) Appl. No.: 15/719,722

(22) Filed: Sep. 29, 2017

Related U.S. Application Data
(63) Continuation of application No. PCT/US2016/025475, filed on Apr. 1, 2016.
(60) Provisional application No. 62/142,088, filed on Apr. 2, 2015.

(51) Int. Cl. C12Q 1/6869; C12Q 1/6806

(52) U.S. Cl. CPC C12Q 1/6869; C12Q 1/6806

(57) ABSTRACT

The invention described herein provides methods and systems for comprehensive genomic analysis that enables the detection of a broad range of genomic variations, including single nucleotide polymorphisms (SNPs), small insertions or deletions (indels), Tandem Base Mutations (TBM), copy number variations (CNVs), structural variations (SVs), and combination thereof, in a single assay.

The invention can be used, for example, to analyze the complicated underlying genomic defects in diseases and conditions such as Autism spectrum disorders (ASD), cancers, Alzheimer's disease, and other neurological disorders.

### REFERENCE TO RELATED APPLICATION

This is a continuation application of International Patent Application No. PCT/US2016/025475, filed on Apr. 1, 2016, which claims the benefit of the filing date of U.S. Provisional Patent Application No. 62/142,088, filed on Apr. 2, 2015, the entire content of each of which, including all drawings and sequence listing (if any), is incorporated herein by reference.

### BACKGROUND OF THE INVENTION

It is known that genetic variations take place in all levels from single nucleotide substitution to large scale structural variation in human population. Many of the genomic variations represent normal phenotypic variation of diverse human traits, whereas some of the variations are linked to diseases. However, the detection and characterization of disease-related genetic variations have been technically challenging, particularly in complex diseases including Autism.

Autism spectrum disorders (ASD) are neurodevelopmental diseases characterized by difficulties or deficits in communication and social interactions. Rates of ASD diagnoses have risen sharply since 2000, from approximately 1 in 150 children to 1 in 68 in 2014 according to the CDC. Diagnostic criteria cover a broad range of symptoms, including behavior and severity of impairment, and patients are often diagnosed with other neuropsychiatric disorders, such as epilepsy. Until recently, the underlying disease pathways for almost all cases of ASD were unknown.

Recent research has shown that ASD and related disorders can be associated with de novo or rare genetic variations that take the form of either large chromosomal alterations or single nucleotide variants (SNV) (Carter and Scherer, Clin. Gen., 83:399-407, 2013; Jiang et al., Am. J. Hum. Gen., 93:249-263, 2013; Pinto et al., Am. J. Hum. Gen. 94:677-694, 2014; Rosti et al., Dev. Med. and Child Neurol., 56:12-18, 2014). Current diagnostic tools include array Comparative Genome Hybridization (aCGH), which identifies copy number variations (CNV)—chromosomal deletions and duplications—in patient DNA. More recently, assays have been developed to identify specific single nucleotide variations (SNV) and small insertions and deletions (indels) in about fifty different genes that are associated with ASD (gene panel tests).

However, aCGH and gene panel tests have to be run separately using different and incompatible technologies (e.g., DNA hybridization vs. DNA sequencing). In addition, the existing gene panel test is limited by the known or potential connections between certain genes and the disease or condition of interest (e.g., ASD), and does not necessarily represent a comprehensive and unbiased approach capable of identifying such small mutations in all relevant genes with known or yet unknown linkage to the disease or condition of interest.

For example, it was recently discovered that children with ASD and macrocephaly may harbor mutations in the PTEN gene. Mutations in PTEN also lead to a dramatic increase in risk of numerous types of cancers including thyroid, breast and skin. Thus children identified as carrying mutations in PTEN require cancer screening beginning in early childhood, as prompt identification of tumors is essential to improving prognosis. Mutations in other autism risk genes, such as POLG, impact risk for toxicity from medications such as valproic acid. Indeed, identification of those at risk is crucial to minimize adverse reactions in this population.

Furthermore, many more genes have recently become associated with ASD but not yet incorporated into currently offered gene sequencing panels. For example, it has just been shown that a mutation in KCNQ2 (Jiang et al., 2013) is associated with autism and may ultimately serve as one target for future personalized treatment, which suggests that Kv7 channel openers may ultimately serve as one target for future personalized treatment of autism (Rundfeldt and Netzer, 2000). This gene, however, is not on any currently available gene panel tests.

The recent advance of high throughput DNA sequencing technology can be adapted for whole genome analysis for ASD and other patients. A possible strategy is to do whole genome shotgun or exome sequencing to identify all SNPs, and a long fragment paired-end-tag sequencing to identify all SVs of a patient's genome. The combination of these approaches will be able to identify all genetic variations. However, it will involve multiple experiments and analysis pipelines, which will be time and resource consuming.

An ideal strategy will be of constructing a single DNA library from one patient sample and conducting a single sequencing run to generate the necessary data for genic SNP calls (currently done by gene panel sequencing), CNV (currently done by aCGH) and SVs (currently done by large fragment PET sequencing) identification in one data analysis pipeline.

Thus a new technology that combines the capabilities of identifying CNVs by aCGH or sequencing with that of limited, targeted sequencing platforms, into a single assay that will be more efficient (time-wise and cost-wise) and comprehensive, could become the new standard of care for ASD molecular diagnoses.

### SUMMARY OF THE INVENTION

The methods and reagents of the invention described herein provide a whole genome analysis technology that enables the detection of a broad range of genomic variations in a host genome (including but are not limited to human ASD patients) in a single assay.

The methods of the invention identifies small and large genomic variations, including SNVs, micro-indels, CNVs, and other large scale genomic structural variations (SVs) such as inversion, tandem duplication, transversions and translocations, all in one unified assay. Many of these large scale genomic structural variations cannot be identified by aCGH or targeted sequencing panels, although they may be detectable by yet other classical cytogenetic banding techniques which are labor intensive.

The clinical utility of the invention described herein has the potential to replace the traditional aCGH and gene panel tests, and promote the emergence of a new standard of care for molecular diagnosis of genetic diseases such as ASD, cancer, and any of many hereditary genetic disorders. In addition, the methods of the invention produces a much richer data set that will have utility for patients as well as translational research.

For example, clinical and genetic data obtained using the methods of the invention can be used to identify at-risk infants, predict clinical outcomes, and develop novel therapeutic regimens for diseases and conditions such as ASD and cancer. Clinical patient data, as well as data generated from the methods of the invention can also be stored in an electronic and/or online database that can serve as a merged, comprehensive, searchable repository of relevant clinical and genetic information. Such database may further include patient baseline information, including but not limited to demographics, patient and family history, presence of co-morbidities, and pertinent physical findings including dysmorphic features, etc. Results of microarray and any other genetic or metabolic testing data can also be added to the database, along with functional and behavioral assessments and results of MRI and EEG, if available/applicable. Unique patient identifiers can be used as matching criteria to enable the results of external analyses to be included within the study database.

Data management for the database may be facilitated by an HIPAA-compliant accessioning database and the Clarity LIMS (Genologics, Vancouver, BC) that tracks the sample and associated quality control (QC) data, as well as the ability to launch an automated bioinformatics workflow.

Thus in one aspect, the invention provides a method for detecting genomic variations in the genome of an organism, the method comprising: (1) fragmenting genomic DNA of the organism to generate a plurality of genomic DNA fragments; (2) tagging the ends of the genomic DNA fragments with a tag sequence; (3) ligating tagged ends of the genomic DNA fragments, under a condition that promotes blunt-end intramolecular ligation, to generate a plurality of circularized genomic DNA fragments with ligated tag sequence; (4) fragmenting the plurality of circularized genomic DNA fragments by shotgun fragmentation, to generate: (a) a plurality of mate-pair (MP) fragments, each comprising the ligated tag sequence flanked by flanking genomic DNA; and, (b) a plurality of shotgun (SG) fragments; (5) determining the sequences of the MP fragments and the SG fragments; and, (6) identifying said genomic variations in the genome of the organism based on both the sequences of the SG fragments and the sequences of the MP fragments.

In certain embodiments, the genomic variations comprise one or more of: single nucleotide polymorphisms (SNPs); small insertions or deletions (indels); tandem base mutations (TBM); copy number variations (CNVs); structural variations (SVs); and combination thereof.

In certain embodiments, steps (1) and (2) are carried out simultaneously.

In certain embodiments, steps (1) and (2) are effected by transposon-mediated tagmentation. For example, the transposon-mediated tagmentation is carried out by a Tn5 transposase.

In certain embodiments, the plurality of genomic DNA fragments is size-selected prior to step (3). In certain embodiments, genomic DNA fragments of about 4-10 kb, or about 6-8 kb, are size-selected.

In certain embodiments, uncircularized or linear genomic DNA fragments are removed by DNA exonuclease digestion prior to steps (4)-(6).

In certain embodiments, sequences of the MP fragments and the SG fragments are determined separately or simultaneously.

In certain embodiments, the SG fragments have an average size of about 400 bp, 450 bp, or 500 bp. In certain embodiments, the MP fragments have an average size of about 400 bp, 450 bp, or 500 bp.

In certain embodiments, the MP fragments and the SG fragments are isolated from each other before step (5).

In certain other embodiments, the MP fragments and the SG fragments are not isolated from each other before step (5).

In certain embodiments, tagged ends of the genomic DNA fragments are repaired to promote blunt end ligation prior to step (3).

In certain embodiments, step (6) comprises mapping the sequences of the flanking genomic DNA and the sequences of the shotgun fragments to the genomic sequence of the organism.

In certain embodiments, sequences of the genomic DNA is determined by high-throughput sequencing. For example, the high-throughput sequencing may be selected from the group consisting of: single-molecule real-time sequencing; ion semiconductor (Ion Torrent) sequencing; pyrosequencing (454); sequencing by synthesis (Illumina); sequencing by ligation (SOLiD sequencing); polony sequencing; massively parallel signature sequencing (MPSS); DNA nanoball sequencing; single molecule nanopore sequencer; and Heliscope single molecule sequencing.

In certain embodiments, the high-throughput sequencing produces 30-, 40-, 50-, 60-, 70-, 80-, 90-, 100- or more fold of coverage for the flanking genomic DNA and/or the shotgun fragments.

In certain embodiments, the organism is a human, a non-human primate, a mammal, a rodent (rat, mouse, hamster, rabbit), livestock animal (cattle, pig, horse, sheep, goat), a bird (chicken), a reptile, an amphibians (Xenopus), a fish (zebrafish (Danio rerio), puffer fish), an insect (Drosophila, mosquito), a nematode, a parasite, a fungus (yeast, such as S. cerevisae or S. pombe), a plant, a bacterium, or a virus.

In certain embodiments, the organism is a human having a disease or condition selected from the group consisting of: autism (autism spectrum disorder (ASD)), cancer, or hereditary disease.

It should be understood that any embodiments described herein, including those only described in the Example section or only under one aspect of the invention, can be combined with any one or more other embodiments, unless specifically disclaimed or otherwise improper.

### BRIEF DESCRIPTION OF THE DRAWINGS

FIGS. 1A and 1B show representative results of detecting SNP and small indels using the methods of the invention.

FIG. 2 shows representative result of detecting a homozygous deletion (CNV) in patient sample P46107, using the methods of the invention.

FIG. 3 shows representative result of detecting a heterozygous deletion (CNV) in patient sample P46107, using the methods of the invention.

FIG. 4 shows a schematic drawing illustrating the detection of inversion and intra-chromosomal direct forward insertion (both SVs), using the method of the invention.

FIG. 5 shows representative result of detecting inversion (SV) only by the MP sequence data, using the methods of the invention.

FIG. 6 shows representative result of detecting an intra-chromosomal translocation (SV), using the methods of the invention.

FIG. 7 representative result of detecting an inter-chromosomal translocation (SV), using the methods of the invention.

FIG. 8 shows the detection of SV in a complex region on Ch. 17.

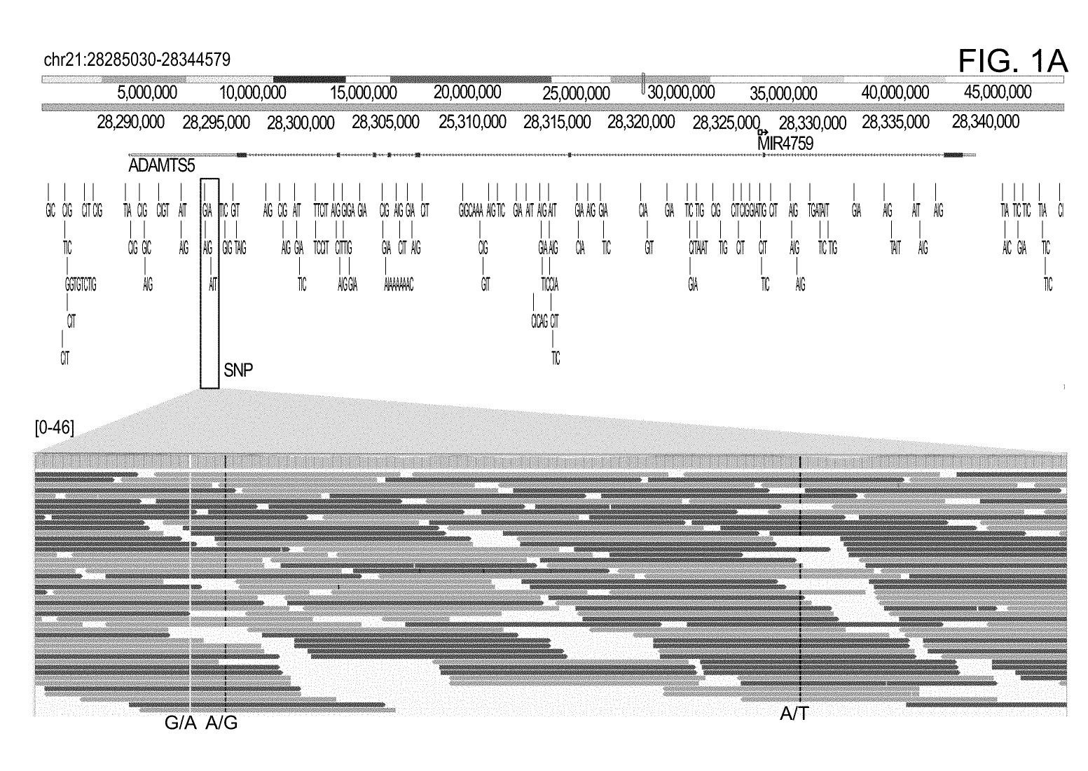

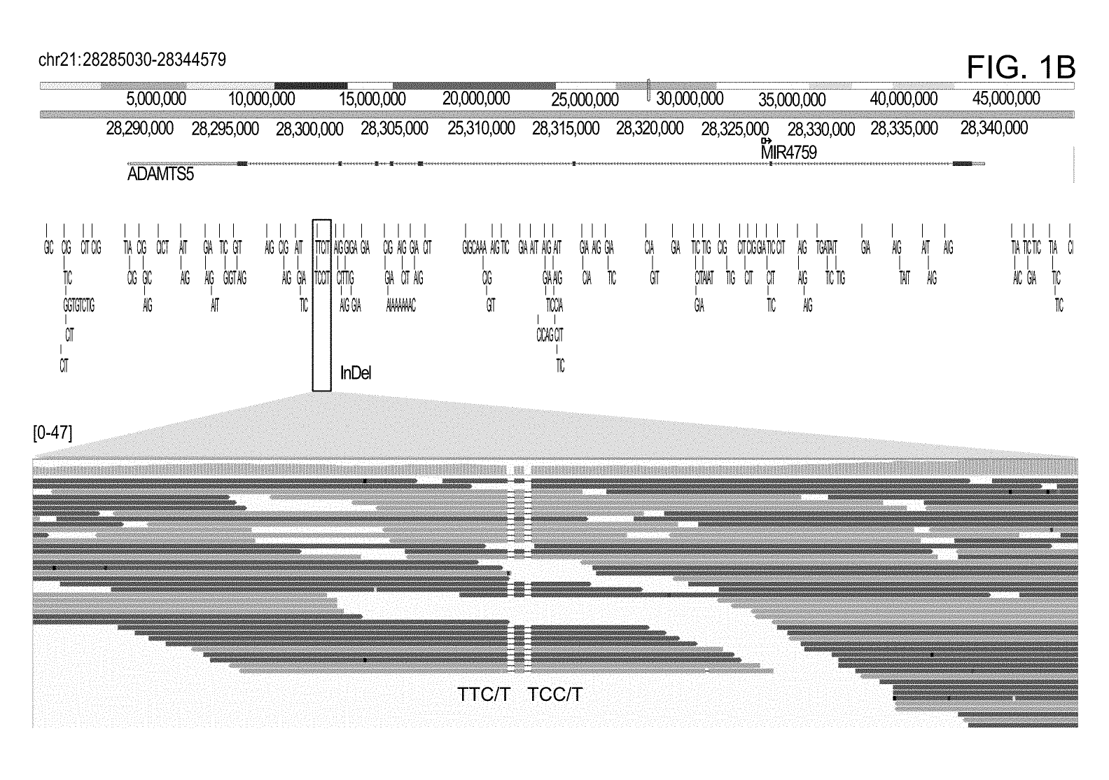

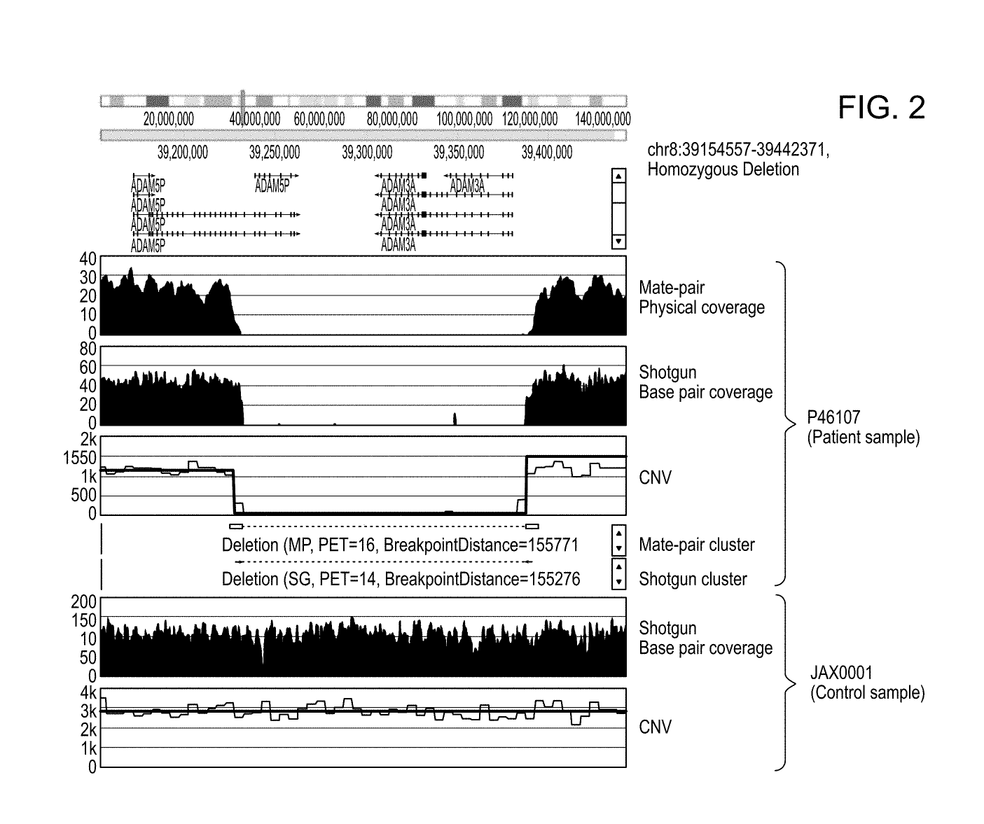

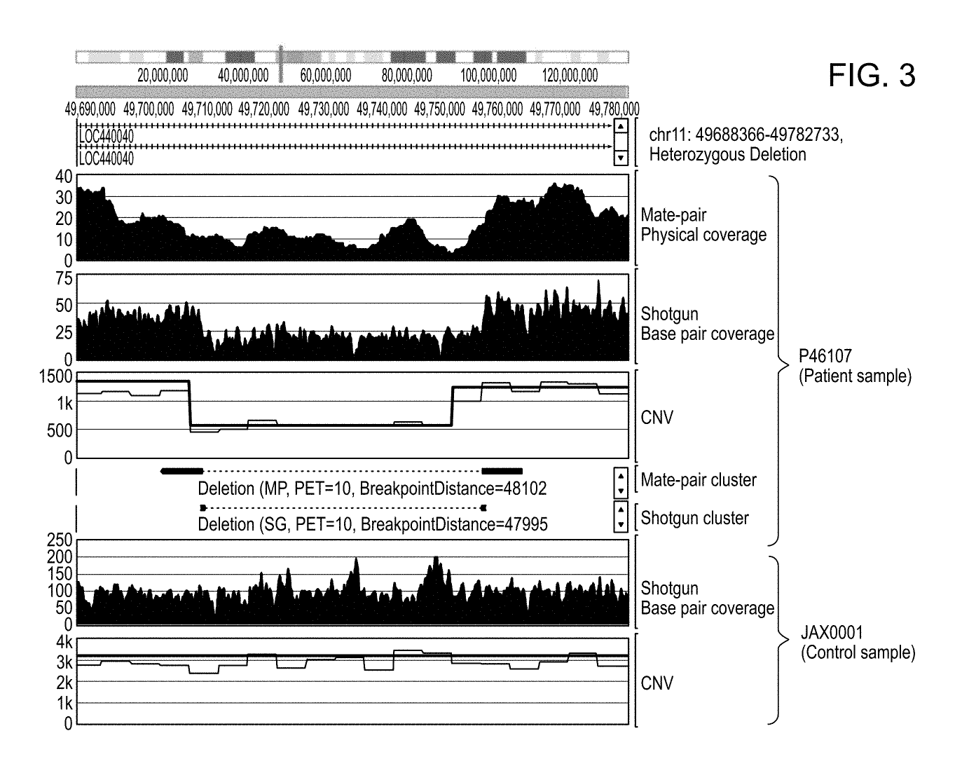

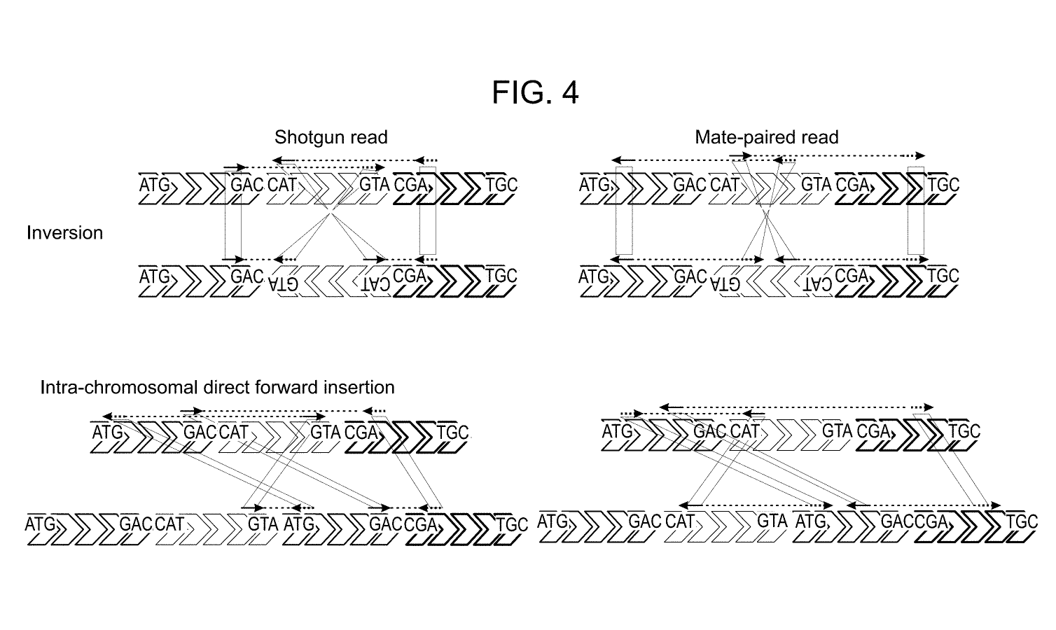

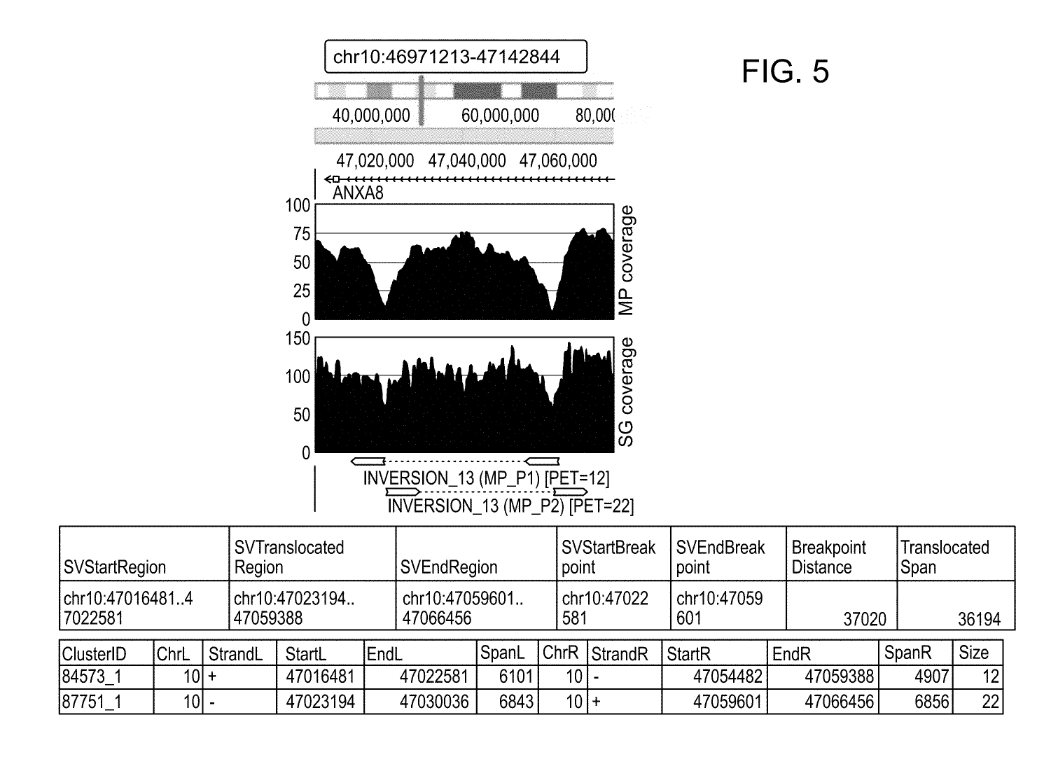

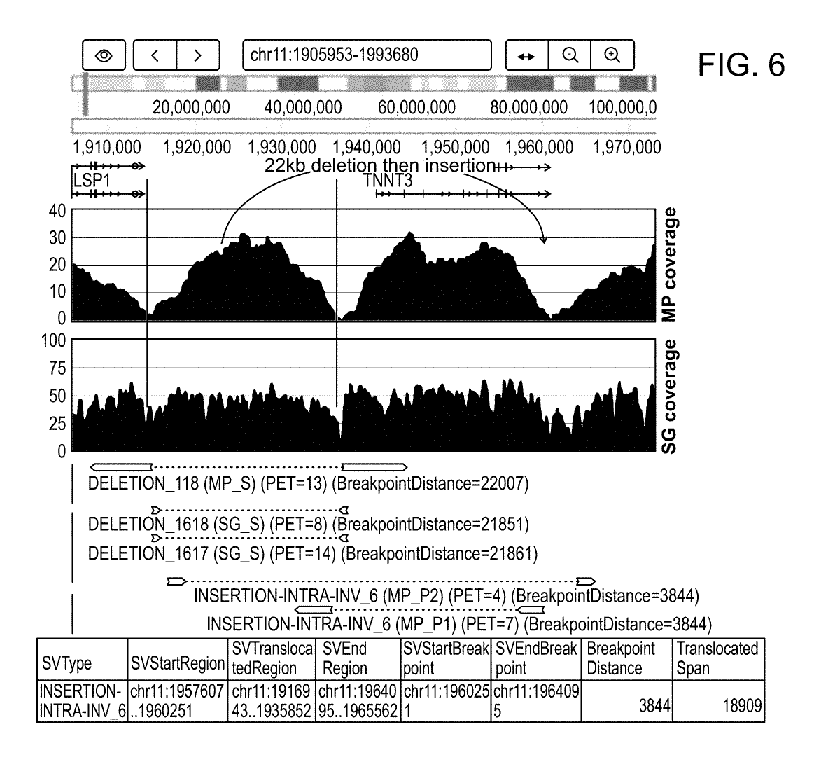

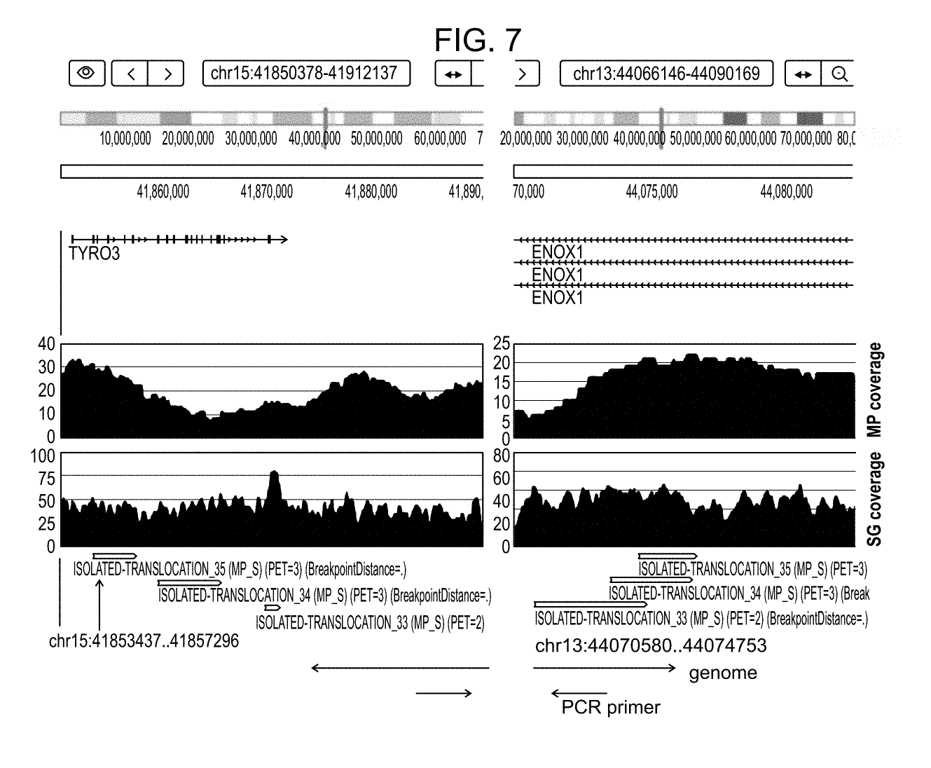

### DETAILED DESCRIPTION OF THE INVENTION

### 1. Overview

The invention described herein provides a fast and efficient means to identify all types of genetic variations from one DNA sample from a patient, through sequencing a uniquely generated genomic DNA library.

Thus in one aspect, the invention provides a method for detecting genomic variations in the genome of an organism, the method comprising: (1) fragmenting genomic DNA of the organism to generate a plurality of genomic DNA fragments; (2) tagging the ends of the genomic DNA fragments with a tag sequence; (3) ligating tagged ends of the genomic DNA fragments, under a condition that promotes blunt-end intramolecular ligation, to generate a plurality of circularized genomic DNA fragments with ligated tag sequence; (4) fragmenting the plurality of circularized genomic DNA fragments by shotgun fragmentation, to generate: (a) a plurality of mate-pair (MP) fragments, each comprising the ligated tag sequence flanked by flanking genomic DNA; and, (b) a plurality of shotgun (SG) fragments; (5) determining the sequences of the MP fragments and the SG fragments; and, (6) identifying said genomic variations in the genome of the organism based on both the sequences of the SG fragments and the sequences of the MP fragments.

Note that the above recited steps do not need to be carried out in the exact order as listed above. Instead, for example, steps (1) and (2) can be carried out simultaneously, in a single step.

The method of the invention can be used to detect genetic variations in any organism, preferably one with a complete or substantially complete genome sequence, including numerous archaeal or eubacterial, protist, fungi (e.g., S. cerevisae or S. pombe), plant, animal genomes. For example, the genome sequences of human, mouse and numerous other mammals and non-mammalian species are now readily available in the public domain. See, for example, Venter et al., “The Sequence of the Human Genome,” Science, 291(5507):1304-1351, 2001. Other non-limiting known genomes include those for numerous non-human primates, mammals, rodents (rats, mice, hamsters, rabbits, etc.), livestock animals (cattle, pigs, horses, sheep, goat), birds (chickens), reptiles, amphibians (Xenopus), fish (zebrafish (Danio rerio), puffer fish), insects (Drosophila, mosquito), nematodes, parasites, fungi (e.g., yeast, such as S. cerevisae or S. pombe), various plants, virus (such as those integrated into a host genome), etc.

In certain embodiments, the organism is a human having a disease or condition selected from the group consisting of: autism (autism spectrum disorder (ASD)), cancer, Alzheimer's disease, other neurological disorders, or hereditary disease or conditions.

The method of the invention can be used to detect numerous types of genetic variations, including but are not limited to: single nucleotide polymorphisms (SNPs); small insertions or deletions (indels); tandem base mutations (TBM); copy number variations (CNVs); structural variations (SVs); or combination thereof. These genetic variations traditionally have to be identified using more than one types of different techniques, almost invariably requiring multiple samples from the patient, or a large sample sufficient to support several runs of different detection methods.

As used herein, single nucleotide polymorphism (SNP) refers to a DNA sequence variation occurring commonly within a population in which a single nucleotide—A, T, C, or G—in the genome (or other shared sequence) differs between members of a biological species or paired chromosomes.

In certain embodiments, the SNP is in a non-coding region of a gene (e.g., transcription enhancer, suppressor, promoter). In another embodiment, the SNP is in a coding region (e.g., open reading frame) of a gene. In yet another embodiment, the SNP is in the intergenic region between two adjacent genes. In certain embodiment, the SNP is in an exon. In certain embodiment, the SNP is in an intron. In certain embodiments, the SNP is in the coding region and represents a silent mutation that does not change the encoded amino acid (synonymous SNP). In a related embodiment, the SNP is in the coding region and is associated with a missense or nonsense mutation (nonsynonymous SNP). In certain embodiments, the SNP occurs in a selected population of a species (e.g., a specific race, ethnic group, religious or faith group of human, or a population confined to a specific geographic location). In certain embodiment, the SNP is associated with a specific disease or condition (e.g., Sickle-cell anemia, (3 Thalassemia, Alzheimer disease, cancer, mandibuloacral dysplasia, progeria syndrome, or Cystic fibrosis), or is indicative of a high risk factor for a disease or condition. In certain embodiment, the SNP is associated with the metabolism of different drugs. In certain embodiments, the SNP is not in a protein-coding region and affects gene splicing, transcription factor binding, messenger RNA degradation, or the sequence of a non-coding RNA (ncRNA). The SNP may be upstream or downstream from the affected gene. In certain embodiments, the SNP is biallelic. In certain embodiments, the SNP is multi-allelic—having 3 or more allelic variations. In certain embodiments, the SNP is any one of the SNPs listed in NCBI's dbSNP (more than 112 million human SNPs as of October 2014). In certain embodiments, the SNP occurs in less than 50%, 40%, 30%, 20%, 10%, 5%, 2%, 1%, 0.5%, 0.2%, 0.1%, 0.05%, 0.01% of a given population (e.g., entire human population, a human population within a country or a geographic location, or a human race, ethnic group, etc.).

As used herein, indel refers to the insertion and/or the deletion of bases in the DNA of an organism, particularly insertion and/or deletion of just a few bases (e.g., 1, 2, 3, 4, 5, 6, 7, 8, 9, 10, 20, 25, 30, 35, 40, 45, 50 etc.). In certain embodiments, the indel does not generate a frame-shift mutation in a coding region. In certain embodiments, the indel does generate a frame-shift mutation or a pre-mature stop codon, or eliminates a natural stop codon.

As used herein, Tandem Base Mutations (TBM) refers to the substitution at adjacent nucleotides, such as substitutions at two adjacent nucleotides, or substitutions at three adjacent nucleotides, etc.

As used herein, copy-number variation (CNV) refers to a form of structural variation in the DNA of a genome that results in the cell having an abnormal or, for certain genes, a normal variation in the number of copies of one or more sections of the DNA. CNVs usually correspond to relatively large regions of the genome that have been deleted (fewer than the normal number) or duplicated/multiplicated (e.g., more than the normal copy number of 2) on certain chromosomes. In certain embodiments, the CNV increases the copy number of a gene. In another embodiment, the CNV reduces the copy number of a gene. In certain embodiments, the genomic region involved in the CNVs is at least about 1 kb, 2 kb, 5 kb, 10 kb, 20 kb, 50 kb, 100 kb, 200 kb, 500 kb, 750 kb, 1 mb, 2 mb, 5 mb or more. In certain embodiments, the CNV is an inherited genetic defect. In another embodiment, the CNV is generated de novo in an individual. In certain embodiments, the CNV can be detected by cytogenetic techniques such as fluorescent in situ hybridization (FISH), comparative genomic hybridization, array comparative genomic hybridization (aCGH), and by virtual karyotyping with SNP arrays. In certain embodiments, the CNV affects a single gene. In another embodiment, the CNV affects two or more genes. In certain embodiment, the CNV has been associated with susceptibility or resistance to a disease or condition (e.g., cancer such as NSCL cancer, SLE, rheumatoid arthritis, inflammatory autoimmune disorder, autism, schizophrenia, or idiopathic learning disability).

As used herein, structural variation (SV, or genomic structural variation) refers to the variation in structure of an organism's chromosome. In a broad sense, SV consists of many kinds of variation in the genome of one species, and usually includes microscopic and submicroscopic types, such as deletions, duplications (such as tandem duplications), copy-number variants, insertions (such as novel sequence insertions and mobile element insertions (MEIs)), inversions, unpaired inversions, and translocations (e.g., isolated vs. balanced translocations). In certain embodiments, SV does not include CNV, or is copy number neutral. In certain embodiments, SV includes inversion, insertion (such as inter-chromosomal direct insertion; inter chromosome inverted insertion; intra-chromosomal direct forward insertion; intra-chromosomal direct backward insertion; intra-chromosomal inverted forward insertion; intrachromosomal inverted backward insertion), translocation, chromosomal rearrangement, ring chromosome, etc., or combination thereof (e.g., deletion plus intra-chromosomal direct forward insertion; deletion plus intra-chromosomal inverted forward insertion.

In certain embodiments, the SV affects a sequence length about 1 kb to 3 Mb, which is larger than SNPs and smaller than chromosome abnormality. Note that the definition of structural variation does not imply anything about frequency or phenotypical effects. In certain embodiments, the structural variant is associated with a genetic diseases or condition. In other embodiments, the structural variation is not associated with any known genetic disease or condition. In certain embodiments, the SV is a microscopic SV that can be detected with optical microscopes, such as aneuploidies, marker chromosome, gross rearrangements, and variation in chromosome size. In certain embodiments, the SV is an inversion, a cryptic translocation, or a segmental uniparental disomy (UPD). In certain embodiments, the SV is listed in a genomic or bioinformatic databases.

In certain embodiments, the genomic variations is in, near, or comprise a region rich in repetitive sequences.

In certain embodiments, the target DNA comprises or consists of a whole genome of a cell or organism. In some embodiments, the target DNA comprises or consists of genomes and/or double-stranded cDNA from multiple organisms (e.g., multiple organisms of the same species, or a representative collection of the organisms) that are present in an environmental sample. In some embodiments, the target DNA comprises or consists of genomes and/or double-stranded cDNA from a specific tissue or organ (e.g., one that is afflicted with a disease or disorder) of an organisms.

In certain embodiments, steps (1) and (2) of the method can be carried out separately. For example, genomic DNA can be fragmented in step (1) using any of many traditional techniques. In one embodiment, DNA fragmentation can be accomplished by physical means, such as acoustic shearing, sonication, or hydrodynamic shearing. Then any desired tag sequences can be ligated to the ends of the fragments. Optionally, the ends of the fragments can be repaired first using DNA polymerases and/or exonucleases to create blunt ends suitable for blunt end ligation.

As used herein, a “tag” or “tag sequence” refers to a non-target nucleic acid, generally DNA, that provides a means of addressing a nucleic acid fragment to which it is joined. For example, in some embodiments, a tag comprises a nucleotide sequence that permits identification, recognition, and/or molecular or biochemical manipulation of the DNA to which the tag is attached (e.g., by providing a site for annealing an oligonucleotide, such as a primer for extension by a DNA polymerase, or an oligonucleotide for capture or for a ligation reaction). The process of joining the tag to the DNA molecule is sometimes referred to herein as “tagging” and DNA that undergoes tagging or that contains a tag is referred to as “tagged” (e.g., “tagged DNA”).

Acoustic shearing and sonication are the main physical methods used to shear DNA, and can be performed using commercially available instruments. For example, the COVARIS® instrument (Woburn, Mass.) is an acoustic device that can fragment DNA into 100 bp-5 kb size range. Covaris also manufactures tubes (gTubes) which can be used to process samples in the 6-20 kb for the subject Mate-Pair libraries. The BIORUPTOR® (Denville, N.J.) is a sonication device suitable for shearing chromatin and DNA to produce genomic fragments of up to 1 kb in length. Hydroshear from Digilab (Marlborough, Mass.) uses hydrodynamic forces to shear DNA. Nebulizers (Life Tech, Grand Island, N.Y.) can also be used to atomize liquid using compressed air, shearing DNA into 100 bp-3 kb fragments in seconds.

In certain embodiments, genomic DNA fragmentation is accomplished by enzymatic means, such as DNase I or other restriction endonuclease or non-specific nuclease, or by Transposase. Enzymatic methods to shear DNA into small pieces include DNAse I, a combination of maltose binding protein (MBP)-T7 Endo I and a non-specific nuclease Vibrio vulnificus (Vvn), NEB's (Ipswich, Mass.) Fragmentase and Nextera tagmentation technology (Illumina, San Diego, Calif.). The combination of non-specific nuclease and T7 Endo synergistically work to produce non-specific nicks and counter nicks, generating fragments that disassociate 8 nucleotides or less from the nick site.

On the other hand, tagmentation uses a transposase to simultaneously fragment and insert transposon ends or transposon end compositions comprising transferred strands (e.g., tag sequences or adaptors) onto dsDNA such as genomic DNA, thus carrying out steps (1) and (2) of the methods simultaneously in a single step. See, for example, WO2010-048605A1 (entire content incorporated herein by reference).

As used herein, a “transposase” is an enzyme that is capable of forming a functional complex with a transposon end-containing composition (e.g., transposons, transposon ends, transposon end compositions) and catalyzing insertion or transposition of the transposon end-containing composition into the double-stranded target DNA with which it is incubated in an in vitro transposition reaction.

A “transposon end” refers to a double-stranded DNA that exhibits only the nucleotide sequences (the “transposon end sequences”) that are necessary to form the complex with the transposase or integrase enzyme that is functional in an in vitro transposition reaction. A transposon end forms a “complex” or a “synaptic complex” or a “transposome complex” or a “transposome composition” with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a “transferred transposon end sequence” or “transferred strand” and a “non-transferred transposon end sequence,” or “non transferred strand.” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that exhibits a “transferred transposon end sequence” (see SEQ ID NO:1 of WO2010048605, incorporated herein by reference), and a non-transferred strand that exhibits a “non-transferred transposon end sequence” (see SEQ ID NO:2 of WO2010048605, incorporated herein by reference).

The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure.

A “transposon end composition” means a composition comprising a transposon end (i.e., the minimum double-stranded DNA segment that is capable of acting with a transposase to undergo a transposition reaction), optionally plus additional sequence or sequences. 5′-of the transferred transposon end sequence and/or 3′-of the non-transferred transposon end sequence. For example, a transposon end attached to a tag is a “transposon end composition.” In some embodiments, the transposon end composition comprises or consists of two transposon end oligonucleotides consisting of the “transferred transposon end oligonucleotide” or “transferred strand” and the “non-transferred strand end oligonucleotide,” or “non-transferred strand” which, in combination, exhibit the sequences of the transposon end, and in which one or both strand comprise additional sequence.

The terms “transferred transposon end oligonucleotide” and “transferred strand” are used interchangeably and refer to the transferred portion of both “transposon ends” and “transposon end compositions,” i.e., regardless of whether the transposon end is attached to a tag or other moiety. Similarly, the terms “non-transferred transposon end oligonucleotide” and “non-transferred strand” are used interchangeably and refer to the non-transferred portion of both “transposon ends” and “transposon end compositions.”

In some embodiments, the transposome is a complex of a wild-type or hyperactive mutant form of a transposase selected from among Tn5 transposase, MuA transposase, Sleeping Beauty transposase, Mariner transposase, Tn7 transposase, Tn10 transposase, Ty1 transposase, and Tn552 transposase and a transposon end with which the transposase forms a complex that is active in a transposition reaction.

In some embodiments, the transposase is a Mu transposase that utilizes transposon ends comprising Mu transposon ends (e.g., HYPERMU™ MuA transposase, EPICENTRE Biotechnologies, Madison, Wis.). In some embodiments, the 3′ portions of the transferred strands comprise a sequence from a Mu transposon end, and wherein the 5′ portions of the transferred strands are not from a Mu transposon.

In some embodiments, the transposase is Tn5 transposase that utilizes transposon ends comprising Tn5 transposon ends (e.g., wild-type or mutant Tn5 transposase, e.g., EZ-Tn5™ transposase, EPICENTRE Biotechnologies, Madison, Wis.). In some embodiments, the 3′ portions of the transferred strands comprise a sequence from a Tn5 transposon end, and wherein the 5′ portions of the transferred strands are not from a Tn5 transposon.

Tagmentation is a modified transposition reaction that takes advantage of the fact that transoposomes randomly insert small free DNA ends (transposon ends or transposon ends composition comprising a transferred strand that has a tag domain in its 5′ portion) into target dsDNA (e.g., genomic DNA), such that the target dsDNA is fragmented to generate a plurality of target dsDNA fragments and a transferred strand of the transposon end or transposon end composition joined to the 5′ ends of each of a plurality of the target dsDNA fragments, and produce a plurality of 5′ tagged target DNA fragments. In certain embodiments, the methods may further comprise incubating the 5′ tagged target DNA fragment with a nucleic acid modifying enzyme under conditions wherein a 3′ tag is joined to a 3′ end of the 5′ tagged target DNA fragment to produce a di-tagged target DNA fragment. The methods are not limited to the use of any particular nucleic acid modifying enzyme. For example, nucleic acid modifying enzymes may comprise polymerases, nucleases, ligases, and the like. In some embodiments, the nucleic acid modifying enzyme comprises a DNA polymerase, and the 3′ tag is formed by extension of the 3′ end of the 5′ tagged target DNA fragment.

In other words, tagmentation effectively fragments the target dsDNA while simultaneously adding on a tag/adaptor/linker sequence that can comprise, for example, PCR primer sites, sequencing primer sites, and/or other moieties that may facilitate isolation or purification of the tagged genomic DNA.

In some embodiments, the tag sequence comprises one or more of a restriction site domain, a capture tag domain, a sequencing tag domain, an amplification tag domain, a detection tag domain, an address tag domain, and/or a transcription promoter domain.

As used herein, a “capture tag domain” or a “capture tag” means a tag domain that exhibits a sequence for the purpose of facilitating capture of the DNA fragment to which the tag domain is joined (e.g., to provide an annealing site or an affinity tag for capturing the tagged DNA fragments on a bead or other surface, e.g., wherein the annealing site of the tag domain sequence permits capture by annealing to a specific sequence which is on a surface, such as a probe on a bead or on a microchip or microarray or on a sequencing bead). In some embodiments, the capture tag domain comprises a 5′-portion of the transferred strand that is joined to a chemical group or moiety that comprises or consists of an affinity binding molecule (e.g., wherein the 5′-portion of the transferred strand is joined to a first affinity binding molecule, such as biotin, streptavidin, an antigen, or an antibody that binds the antigen, that permits capture of the tagged DNA fragments on a surface to which a second affinity binding molecule is attached that forms a specific binding pair with the first affinity binding molecule).

For example, the tag sequence used by the transposome may comprise a biotinylated junction adaptor such that the tagged genomic fragments can be isolated using streptavidin beads.

As used herein, a “sequencing tag domain” or a “sequencing tag” means a tag domain that exhibits a sequence for the purposes of facilitating sequencing of the DNA fragment to which the tag is joined (e.g., to provide a priming site for sequencing by synthesis, or to provide annealing sites for sequencing by ligation, or to provide annealing sites for sequencing by hybridization).

In some embodiments, the sequencing tag domains comprise or consist of sequencing tags selected from Roche 454A and 454B sequencing tags, ILLUMINA™ SOLEXA™ sequencing tags, Applied Biosystems' SOLID™ sequencing tags, the Pacific Biosciences' SMRT™ sequencing tags, Pollonator Polony sequencing tags, or the Complete Genomics sequencing tags.

As used herein, an “amplification tag domain” means a tag domain that exhibits a sequence for the purpose of facilitating amplification of a nucleic acid to which said tag is appended. For example, in some embodiments, the amplification tag domain provides a priming site for a nucleic acid amplification reaction using a DNA polymerase (e.g., a PCR amplification reaction or a strand-displacement amplification reaction, or a rolling circle amplification reaction), or a ligation template for ligation of probes using a template-dependent ligase in a nucleic acid amplification reaction (e.g., a ligation chain reaction).

In some embodiments, the methods further comprise amplifying one or more tagged target DNA fragments and/or di-tagged target DNA fragments. In some embodiments, the amplifying comprises use of one or more of a PCR amplification reaction, a strand-displacement amplification reaction, a rolling circle amplification reaction, a ligase chain reaction, a transcription-mediated amplification reaction, or a loop-mediated amplification reaction. In certain embodiments, amplifying comprises non-selectively amplifying tagged target DNA fragments of a DNA fragment library or di-tagged target DNA fragments of a DNA fragment library.

As used herein, an “address tag domain” or an “address tag” means a tag domain that exhibits a sequence that permits identification of a specific sample (e.g., wherein the transferred strand has a different address tag domain that exhibits a different sequence for each sample).

Two transposomes can be mixed in equimolar ratios, each carrying one of the two small free DNA ends that encompasses PCR/sequencing sites. That is, in some of embodiments, the method comprises simultaneously incubating the target DNA with both a first transposase and a first transposon end oligonucleotides and a second transposase and a second transposon end oligonucleotides in the same reaction mixture. In some other embodiments, the method is performed sequentially by first incubating the target DNA with the first transposase and the first transposon end oligonucleotides and then incubating the products from that reaction with the second transposase and the second transposon end oligonucleotides. In some of the embodiments wherein the method is performed sequentially, the products from the reaction of the target DNA with the first transposase and the first transposon end oligonucleotides are purified before incubating those products with the second transposase and the second transposon end oligonucleotides.

In some embodiments, the transposon end composition used on tagging a fragment or library comprises a plurality of transferred strands that differ in nucleic acid sequence by at least one nucleotide, and the amplifying comprises selectively amplifying di-tagged DNA fragments based on the nucleic acid sequences of the 5′ end tags or tag domains. In other embodiments, the amplifying comprises a PCR using a single oligonucleotide primer that is complementary to the 3′ tag of the di-tagged target DNA fragments.

In some embodiments, the amplifying comprises a strand-displacement amplification reaction using a single oligonucleotide primer, in which the oligonucleotide primer consists of only ribonucleotides, or consists of only purine ribonucleotides and only pyrimidine 2′-F-2′-deoxyribonucleotides, and the strand displacement amplification reaction comprises a strand-displacing DNA polymerase and a ribonuclease H.

In some embodiments, the amplifying comprises a polymerase chain reaction using a first and a second oligonucleotide primer, each comprising 3′ end portions, wherein at least the 3′ end portion of the first PCR primer is complementary to the 3′ tag of the di-tagged target DNA fragments, and wherein at least a the 3′-end portion of the second PCR primer exhibits the sequence of at least a portion of the 5′ tag or tag domain of the di-tagged target DNA fragments. In certain embodiments, the first or second oligonucleotide primer comprises a 5′ end portion, wherein at least the 5′ end portion of the first primer is not complementary to the 3′ tag of the di-tagged target DNA fragments, or wherein the 5′ portion of the second primer does not exhibit the sequence of at least a portion of the 5′ tag or tag domain of the di-tagged target DNA fragments. In certain embodiments, the first and a second oligonucleotide primers each comprise 5′ end portions, wherein at least the 5′ end portion of the first PCR primer is not complementary to the 3′ tag of the di-tagged target DNA fragments, and/or wherein the 5′-end portion of the second PCR primer does not exhibit the sequence of at least a portion of the 5′ tag domain of the di-tagged target DNA fragments.

In some embodiments, it is useful to amplify the fragments and libraries of the invention. Thus, in some embodiments, the amplifying comprises a polymerase chain reaction using a first and a second oligonucleotide primer, each comprising 3′ end portions complementary to at least a portion of one sequence of the transferred strand in the tagged DNA fragments or in the di-tagged DNA fragments.

Since each transposome can only tagment once, the average size of the fragments is primarily determined by the ratio of input genomic DNA to transposomes.

Thus in certain embodiments, the amount of the input genomic DNA is accurately determined, for example, by using a method that specifically quantitates the amount of dsDNA in a sample, or a method that avoids detecting contaminating RNA, ssDNA, or degraded DNA in a sample. Commercial products, such as QUBIT® assays (Life Technologies, Thermo Fisher Scientific, Inc.) can be used for this purpose, and the results can be read in the QUBIT® Fluorometer.

In certain embodiments, the average size of the tagmented genomic DNA is about 4, 5, 6, 7, 8, 9, 10, 11, 12, 13, 14, or 15 kb. In certain embodiments, the average size of the tagmented genomic DNA is about 4-10 kb, or about 6-8 kb.

In certain embodiments, the ends of the fragmented and tagged DNA fragments have single-stranded regions that are preferably filled in or repaired prior to the next step. That is, in certain embodiments, tagged ends of the genomic DNA fragments are repaired to promote blunt end ligation prior to step (3). This may be necessary for fragments generated using transposome-mediated tagmentation, since the tagmentation step leaves a short single stranded sequence gap in the tagmented DNA. In such embodiments, polymerase-mediated strand displacement reaction can be used to fill in the gap created by the tagmentation step to ensure that all fragments are flush.

In some embodiments, the filling and ligating steps comprise incubating the tagged DNA fragments with the one or more sizes of random-sequence oligonucleotides and the template-dependent ligase under conditions wherein the random-sequence oligonucleotides anneal and fill single-stranded gaps and are ligated to each other or to adjacent ends of tagged DNA fragments.

In certain embodiments, the fragmented or tagmented DNA is size-selected prior to step (3). In certain embodiments, one pre-determined size of fragmented or tagmented DNA is size-selected for use in the subsequent steps, e.g., circularization of the size-selected DNA. In certain embodiments, two or more different pre-determined sizes of fragmented or tagmented DNA is size-selected, each size of selected DNA circularized and used together in the further shotgun fragmentation steps. If more than one sizes are selected, each size can be distinguished from the other via, for example, the different tag sequences used to generate end-tagged genomic DNA fragments.

Any of many art recognized methods can be used for DNA size selection. In one embodiment, size selection is carried out by PEG (polyethylene glycol)-mediated DNA precipitation. See, for example, Lis and Schleif, “Size Fractionation of Double-Stranded DNA by Precipitation with Polyethylene Glycol,” Nuc. Acid Res., 2(3):383-389 (1975). Entire content incorporated herein by reference. In particular, at lower PEG concentration, large dsDNA precipitates better than smaller dsDNA (e.g., those <1500 bp). Using this method, it was reported that size fractionation can be achieved for DNA in the size range of about 150 bp-50 kb. In certain embodiments, PEG-mediated size selection is regulated by varying PEG concentration, DNA concentration, NaCl concentration, pH, divalent ions, precipitation time, and/or centrifugation forces.

Commercial products are readily available to facilitate PEG precipitation-based size selection, such as Agencourt AMPure XP bead (BD, see, for example, Item Number A63880) or SPRIselect bead (BD, see, for example, Item Number B23317). Larger DNA fragments are bound by those beads, while smaller fragments (e.g., those <1500 bp) remain in solution and are readily removed.

In another embodiment, size selection is carried out by agarose-gel electrophoresis. For example, the Pippin DNA Size-Selection System (Sage Science) is an automated preparative agarose gel electrophoresis system that can select a specified size range of DNA sample. The BLUEPIPPIN™ system can be used to collect DNA within a narrow distribution size, ranging between 90 bp to 50 kb, according to the manufacturer. Similarly, the PIPPINPREP™ system can be used to collect DNA fragments of 90 bp-8 kb. In certain embodiments, DNA fragments with an average size between 1-50 kb, such as 6-8 kb or 4-10 kb is size-selected using about 0.75% agarose in a BLUEPIPPIN™ type system. In certain embodiments, DNA fragments with an average size between 2-8 kb is size-selected using about 0.75% agarose in a PIPPINPREP™ type system. In certain embodiments, the collected DNA has a narrow size distribution of ±3 kb, 2 kb, 1 kb, or 0.5 kb.

In certain embodiments, standard agarose gel electrophoresis can also be used without the Pippin DNA Size-Selection System, especially when several size ranges are to be selected from one run. The size-selected DNA fragment can be recovered or purified from the gel using any art-recognized methods. In one embodiment, the DNA is recovered by spin column-based DNA recovery reagents, such as the commercially available ZYMOCLEAN™ Large Fragment DNA Recovery Kit (Zymo Research).

In certain embodiments, one or more of the above size-selection methods can be used in combination, such as PEG precipitation-based size selection followed by agarose-gel electrophoresis-based size selection.

Once the tagged DNA fragment is obtained, preferably within a pre-determined size range, the ends of the fragment are ligated under a condition that promotes or favors blunt-end intramolecular ligation, to generate a plurality of circularized genomic DNA fragments. In certain embodiments, the condition comprises ligating DNA fragments in relatively large volume and low concentration, such as 0.05-0.2 ng/μL (e.g., about 0.1 ng/μL), or 1.5-3 ng/μL (e.g., about 2 ng/μL), of 6-8 kb size-selected DNA. The ligation may be carried out overnight (e.g., 12-16 hrs), at the optimum temperature of the DNA ligase (e.g., 30° C.).

In some embodiments, the method further comprises separating the tagged circular DNA fragments from linear DNA, unligated random sequence oligonucleotides, and/or transposon end composition not joined to target DNA.

In certain embodiments, unligated linear DNA is removed by DNA exonuclease. For example, in some embodiments, the reaction mixture containing the tagged circular DNA fragments is treated with T5 exonuclease to remove linear DNA, such as unligated fragments and random-sequence oligonucleotides.

In certain embodiments, the circularized genomic DNA fragments is fragmented again by shotgun fragmentation to generate a plurality of smaller fragments, which is generally in a size-range suitable for sequencing. For example, fragments of about 300-1000 bp (e.g., 400, 450, or 500 bp) can be generated for any of the art-recognized sequencing methods, such as one of the many next-generation sequencing (NGS) methods.

The same acoustic shearing and sonication methods can be used for shotgun fragmentation. For example, the COVARIS® instrument (Woburn, Mass.) can be used to generate DNA fragments of about 300-1000 bp (e.g., 400, 450, or 500 bp). Alternatively, in another embodiment, shotgun fragmentation is carried out using a nebulizer to produce fragments of about 300-1000 bp.

In certain embodiments, the genomic DNA is fragmented and tagged using transposome-mediated tagmentation, and the tag sequence used in tagmentation comprises a moiety that can facilitate isolation or purification of the tag sequence. For example, the tag sequence can be a biotinylated junction adaptor, which can be isolated by SA-beads. The fragments attached to the SA-beads form a mate-pair (MP) fragment library in which the short genomic DNA fragments contain at least one (usually both) of the tag sequences. That is, the majority of the short genomic DNA fragments are two linked junction adaptors (tag sequences), flanked by two genomic DNA fragments that were separated by many kbs (depending on the average size of the mate-pair library) in the genome. The sequences of the individual fragments in the MP fragment library can be determined using any of art-recognized sequencing methods, such as one of the many NGS methods described below, to produce the MP fragments sequencing data.

The fragments generated by shotgun fragmentation and not bound by the SA-beads, instead of being discarded, can also be collected and sequenced similarly, for example, by NGS, to produce the shotgun fragments sequencing data. Such fragments without the tag sequence are also referred to as shotgun (SG) fragments. In certain embodiments, the SG fragments also include fragments having partial tag sequences, usually at one end of such fragments.

In certain embodiments, the MP fragments and the SG fragments are separated prior to further treatment. Separation can be achieved using any affinity tag in the tag sequence, now only present in the MP fragments but not the SG fragments.

In other embodiments, the MP fragments and SG fragments are processed together, including sequenced together. Sequence data from the MP fragments can be distinguished from that of the SG fragments by the presence (vs. absence) of the tag sequence in the MP fragments. In this embodiment, it is not necessary to use tag sequences that facilitates separation of the MP fragments and the SG fragments.

Both the MP and SG fragments can be optionally repaired by filling in or removing the 5′ or 3′ overhangs that are the result of shotgun fragmentation, in order to create blunt ends. For example, 3′ to 5′ exonuclease activity can be used to remove the 3′ overhangs, and polymerase activity can fill in the 5′ overhangs.

In certain embodiments, a single Adenine nucleotide is added to the 3′ ends of the blunt fragments to prevent them from ligating to one another during a future adapter ligation reaction. A corresponding single Thymidine nucleotide on the 3′ end of the adapter provides a complementary overhang for ligating the adapter to the fragment. This strategy ensures a low rate of chimera (concatenated template) formation.

In certain embodiments, adaptor ligation is performed to ligate any desired adapters to the blunt ends of the DNA fragments, preparing them for, e.g., a future PCR amplification.

The SG and MP DNA fragments can be used as templates in a DNA sequencing method (e.g., NGS) or an amplification reaction prior to sequencing. In some embodiments, the methods of the invention comprise amplifying the MP/SG DNA fragments, e.g., by using of one or more of a PCR amplification reaction, a strand-displacement amplification reaction, a rolling circle amplification reaction, a ligase chain reaction, a transcription-mediated amplification reaction, or a loop-mediated amplification reaction. In some embodiments, the amplifying comprises a polymerase chain reaction using a first and a second oligonucleotide primer, each comprising 3′ end portions, wherein at least the 3′ end portion of the first PCR primer is complementary to at least a portion of the tag domain, and wherein at least a the 3′-end portion of the second PCR primer exhibits the sequence of at least a portion of the tag domain. In some embodiments, the first and second oligonucleotide primers each comprise 5′ end portions, wherein the 5′ end portion of the first PCR primer is not complementary to the tag sequence, and wherein the 5′-end portion of the second PCR primer does not exhibit the sequence of the tag domain.

Preferred embodiments of any of the PCR amplification described above comprise amplifications wherein the 5′ end portions of the first and/or the second PCR primers exhibit tag domains. In still more embodiments, the tag domains comprise one or more of a restriction site domain, a capture tag domain, a sequencing tag domain, an amplification tag domain, a detection tag domain, an address tag domain, and a transcription promoter domain.

In some embodiments, the tag domains are sequencing tag domains that comprise or consist of sequencing tags selected from Roche 454A and 454B sequencing tags, ILLUMINA™ SOLEXA™ sequencing tags, Applied Biosystems' SOLID™ sequencing tags, the Pacific Biosciences' SMRT™ sequencing tags, Pollonator Polony sequencing tags, or the Complete Genomics sequencing tags.

PCR conditions can be adjusted depending on specific needs. A typical PCR condition in a thermal cycler may include: 98° C. for 30 seconds; 10-15 cycles of PCR with 98° C. for 10 seconds, 60° C. for 30 seconds, and 72° C. for 30 seconds; 72° C. for 5 minutes, and hold at 4° C.

In certain embodiments, sequences of the genomic DNA is determined by high-throughput sequencing. “Sequencing” refers to the various methods used to determine the order of constituents in a biopolymer, in this case, a nucleic acid.

Suitable sequencing techniques that can be used with the instant invention includes the traditional chain termination Sanger method, as well as the so-called next-generation (high throughput) sequencing (NGS) available from a number of commercial sources, such as massively parallel signature sequencing (or MPSS, by Lynx Therapeutics/Solexa/Illumina), polony sequencing (Life Technologies), pyrosequencing or “454 sequencing” (454 Life Sciences/Roche Diagnostics), sequencing by ligation (SOLiD sequencing, by Applied Biosystems/Life Technologies), sequencing by synthesis (Solexa/Illumina), DNA nanoball sequencing, heliscope sequencing (Helicos Biosciences), ion semiconductor or Ion Torrent sequencing (Ion Torrent Systems Inc./Life Technologies), and single-molecule real-time (SMRT) sequencing (Pacific Bio), etc. Numerous other high throughput sequencing methods are still being developed or perfected, with may also be used to sequence the MP or SG fragments of the invention, including nanopore DNA sequencing, sequencing by hybridization, sequencing with mass spectrometry, microfluidic Sanger sequencing, transmission electron microscopy DNA sequencing, RNAP sequencing, and In vitro virus high-throughput sequencing, etc.

In certain embodiments, the high-throughput sequencing may be selected from the group consisting of: single-molecule real-time sequencing; ion semiconductor (Ion Torrent) sequencing; pyrosequencing (454); sequencing by synthesis (Illumina); sequencing by ligation (SOLiD sequencing); polony sequencing; massively parallel signature sequencing (MPSS); DNA nanoball sequencing; single molecule nanopore sequencer; and Heliscope single molecule sequencing.

In certain embodiments, the high-throughput sequencing produces 10-, 15-, 20-, 25-, 30-, 40-, 50-, 60-, 70-, 80-, 90-, 100- or more fold of coverage for the flanking genomic DNA and/or the shotgun fragments.

In certain embodiments, the sequencing method is capable of sequencing tag sequences from both ends of the subject tagged genomic DNA fragments, thus providing paired end tag information. In certain embodiments, the sequencing method is capable of performing reads on long DNA fragments of variable length.

Both the MP fragments sequencing data and the SG fragments sequencing data can then be used in the methods of the invention to determine all genetic variations, as elaborated below. In certain embodiments, all sequence data are mapped to a matching reference genome. As used herein, “mapping (a sequence to a genome)” includes the identification of the genomic location of the sequence in the genome.

That is, the methods of the invention rely on sequencing data from both the MP fragments (that represent sequences at the two ends of each long genomic DNA fragment) and the SG fragments without the tag sequence (that represent sequences between the two ends), wherein the MP fragments and the shotgun fragments are from the same library of plurality of circularized genomic DNA fragments.

For example, for a circularized genomic DNA of about 10 kb in size, if the shotgun fragmentation produces fragments of about 500 bp in size, one of the 500 bp fragments is expected to be the Mate-Pair fragment comprising the tag sequence flanked by two ˜200 bp sequences, one from each ends of the 10 kb fragment. Meanwhile, 19 of the 500 bp fragments are expected to be the shotgun fragments without the tag sequence, which represent the 9.5 kb sequences between the two ends. Therefore, on average, one sequencing read from the MP fragment corresponds to about 19 sequencing reads from the shotgun fragment reads. This one to 19 expected ratio is partly depending on the average size of the circularized genomic DNA fragment (e.g., 10 kb), and is partly depending on the average size of the MP and SG fragments generated by shotgun fragmentation (e.g., 500 bp).

Similarly, for CNV type genomic variation, if there is a homozygous deletion in the genome, both the MP fragment sequencing data and the SG fragment sequencing data will reveal a gap on the sequence coverage map when all sequence reads are mapped to the genome of the organism.

On the other hand, for heterozygous deletion in the genome, both the MP fragment sequencing data and the SG fragment sequencing data will exhibit about half the amount of the deleted region as compared to other regions of the genome without deletion.

With the inventions generally described above, certain specific aspects of the invention are further described below.

It is contemplated that any one embodiments of the invention can be combined with any one or more other embodiments of the invention unless inappropriate, inapplicable, or specifically disclaimed.

### 2. Next Generation Sequencing (NGS)

Sequencing of the MP fragments and/or SG fragments can be done using any art recognized methods. In certain embodiments, sequencing is performed using the so-called next generation sequencing (NGS) high throughput sequencing.

Next-generation sequencing platforms that can be used with the methods of the invention include (but are not limited to) the 454 FLX™ or 454 TITANIUM™ (Roche), the SOLEXA™ Genome Analyzer (Illumina), the HELISCOPE™ Single Molecule Sequencer (Helicos Biosciences), and the SOLID™ DNA Sequencer (Life Technologies/Applied Biosystems) instruments), as well as other platforms still under development by companies such as Intelligent Biosystems and Pacific Biosystems.

Although the chemistry by which sequence information is generated varies for the different next-generation sequencing platforms, all of them share the common feature of generating sequence data from a very large number of sequencing templates, on which the sequencing reactions are run simultaneously. In general, the data from all of these sequencing reactions are collected using a scanner, and then assembled and analyzed using computers and powerful bioinformatics programs. The sequencing reactions are performed, read, assembled, and analyzed in a “massively parallel” or “multiplex” fashion. The massively parallel nature of these instruments has resulted in a change as to what kind of sequencing templates are needed and how to generate them in order to obtain the maximum possible amounts of sequencing data from these powerful instruments.

In particular, the NGS sequencing methods utilize DNA fragment libraries generated in vitro and comprising a collection or population of DNA fragments generated from target DNA in a sample, wherein the combination of all of the DNA fragments in the collection or population exhibits sequences that are qualitatively and/or quantitatively representative of the sequence of the target DNA from which the DNA fragments were generated. In fact, DNA fragment libraries consisting of multiple genomic DNA fragment libraries, such as the MP fragment library and the SG fragment library, each of which is labeled with a different address tag or bar code (e.g., with or without the tag sequence or junction adaptor) to permit identification of the source of each fragment sequenced.

In general, these NGS methods require fragmentation of genomic DNA into smaller ssDNA fragments and addition of tag sequences (or “tags” in short) to at least one strand or preferably both strands of the ssDNA fragments. In some methods, the tags provide priming sites for DNA sequencing using a DNA polymerase. In some methods, the tags also provide sites for capturing the fragments onto a surface, such as a bead (e.g., prior to emulsion PCR amplification for some of these methods; e.g., using methods as described in U.S. Pat. No. 7,323,305). In most cases, the DNA fragment libraries used as templates for NGS comprise 5′- and 3′-tagged DNA fragments or “di-tagged DNA fragments.” In general, existing methods for generating DNA fragment libraries for NGS comprise fragmenting the target DNA that one desires to sequence (e.g. target DNA comprising genomic DNA) using a sonicator, nebulizer, or a nuclease, and joining (e.g., by ligation) oligonucleotides consisting of adapters or tags to the 5′ and 3′ ends of the fragments.

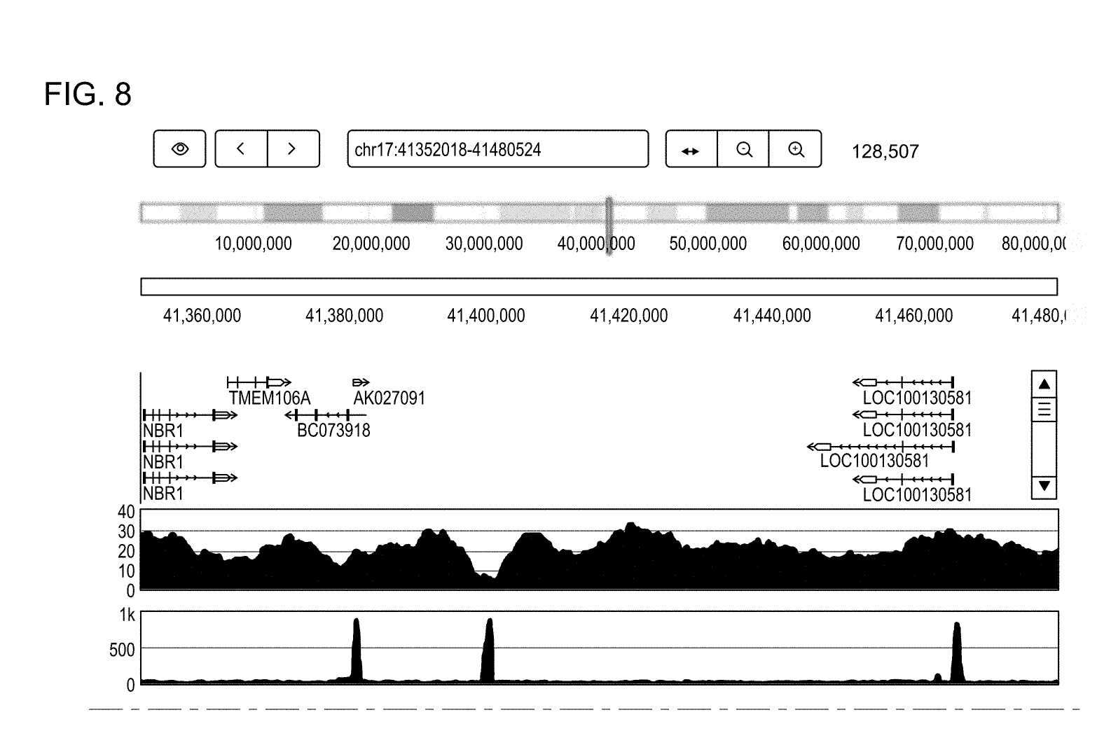

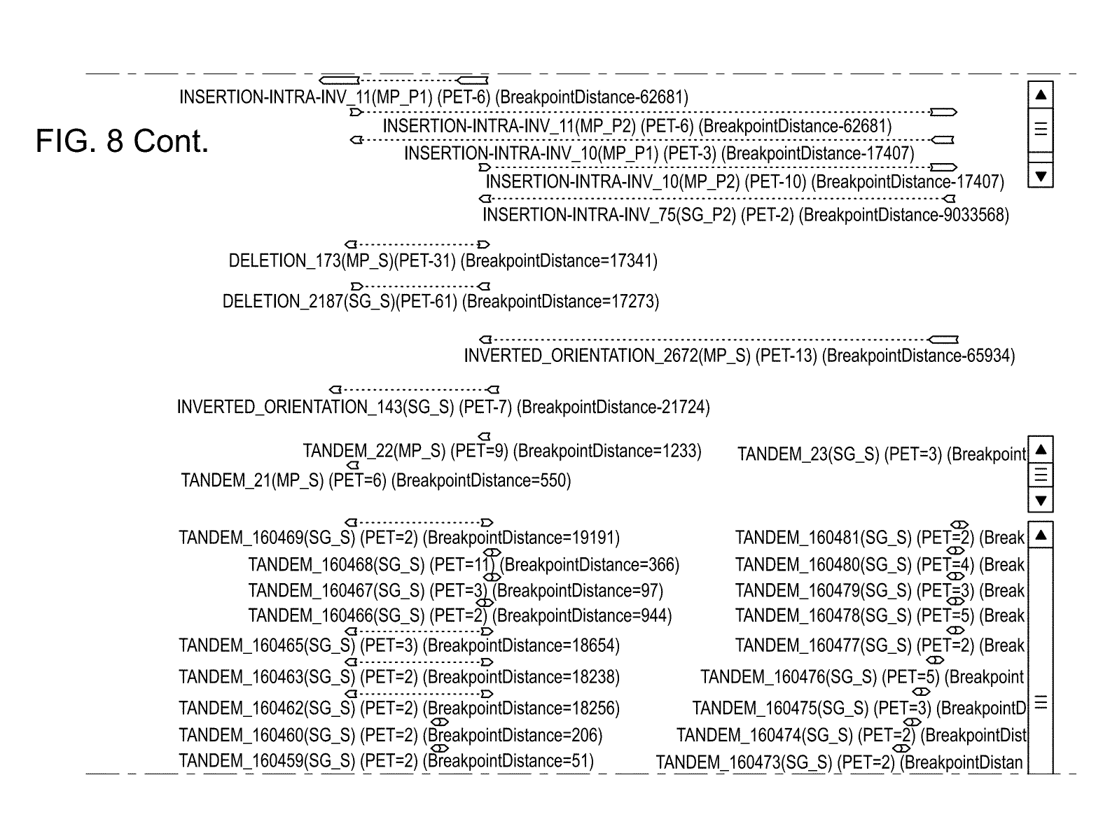

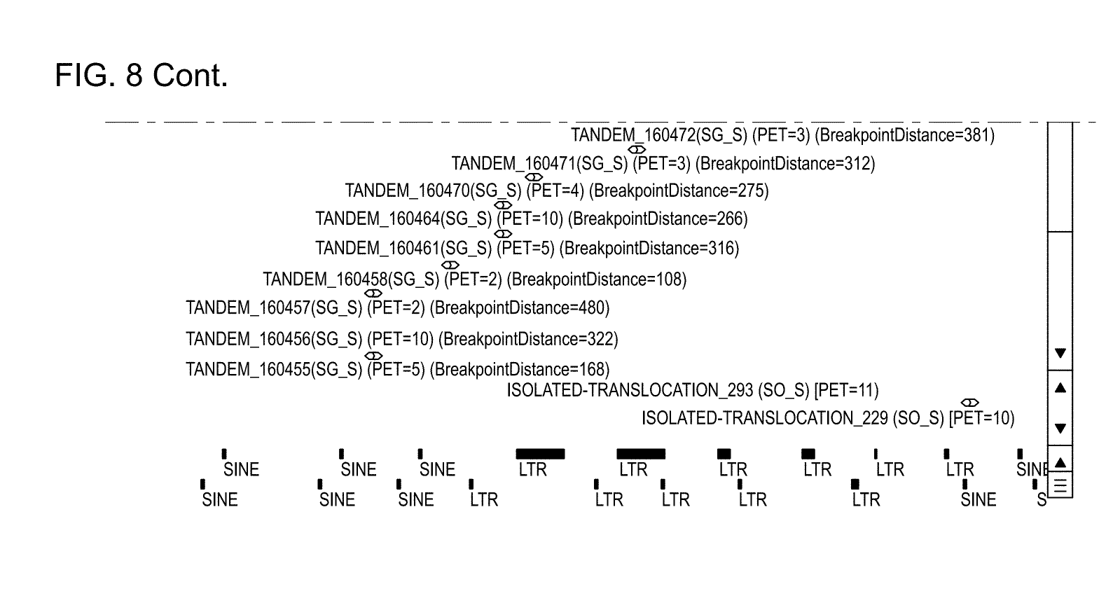

Some of the NGS methods use circular ssDNA substrates in their sequencing process. For example, U.S. Patent Application Nos. 2009-0011943; 2009-0005252; 2008-0318796; 2008-0234136; 2008-0213771; 2007-0099208; and 2007-0072208 of Drmanac et al., each incorporated herein by reference, discloses generation of circular ssDNA templates for massively parallel DNA sequencing. U.S. Patent Application No. 2008-0242560 of Gunderson and Steemers discloses methods comprising: making digital DNA balls (see, e.g., FIG. 8 in U.S. Patent Application No. 2008-0242560); and/or locus-specific cleavage and amplification of DNA, such as genomic DNA, including for amplification by multiple displacement amplification or whole genome amplification (e.g., FIG. 17 therein) or by hyperbranched RCA (e.g., FIG. 18 therein) for generating amplified nucleic acid arrays (e.g., ILLUMINA BeadArrays™; ILLUMINA, San Diego Calif., USA).

Additional NGS methods with amplification, such as whole genome amplification, also require fragmentation and tagging of genomic DNA. Some of these methods are reviewed in: Whole Genome Amplification, ed. by S. Hughs and R. Lasken, 2005, Scion Publishing Ltd. (on the worldwide web at scionpublishing.com), incorporated herein by reference. These NGS methods can also be used in the methods of the invention.

### 3. Sequencing Data Analysis and Detection of Genomic Variations

Once the sequence information is obtained from the SG fragments and the MP fragments through, for example, high throughout sequencing using any of the many applicable NGS methods, the method of the invention provides sequence data analysis to determine the various genomic variations in the genome of the subject.

In one embodiment, sequences for the SG fragments and the MP fragments are obtained simultaneously based on NGS of the products of the shotgun fragmentation. Sequences belonging to the MP fragments can be generally distinguished from those of the SG fragments based on the presence of the ligated tag sequences (e.g., 2 ligated tandem repeats of a 19-base pair tag sequence used in tagmentation) flanked by genomic DNA sequences. The tag sequences can be removed from the raw sequence data to preserve only genomic sequences in the MP fragments. In addition, genomic sequences from the MP fragments can be separately stored, saved, or manipulated in a separate database for data file from that for the SG fragments.

The sequences of the SG fragments and MP fragments can then be mapped to a matching reference genome. For example, the well-characterized human genome sequence can be used as the reference genome for any human samples from a human subject. Other model organism reference genomes are readily available in the art.

In one embodiment, the SG fragment sequences are mapped to the matching reference genome to create a first mapping file, and the MP fragment sequences are mapped to the same matching reference genome to create a second mapping file, for use with the methods of the invention. These mapping files can be generated using any of many art-recognized and publically available mapping software, such as the Burrows-Wheeler Aligner (BWA) developed by Heng Li of the Broad Institute. See, Henry Li, Aligning New-sequencing Reads by BWA (2010), the entire content is incorporated herein by reference.

In general, these sequence aligning software aligns sequencing reads (such as the reads from the NGS methods) against a known reference sequence for variation discovery, while overcoming difficulties such as efficiency and ambiguity caused by sequencing repeats and sequencing errors. Many sequence aligners for long sequence reads (e.g., reads that are over about 200 bp) are available, including BLAT, SSAHA2, and BWA-SW. Numerous short read (for sequences about or less than 100 bp) aligners are also available, including but are not limited to: Bfast, BioScope, Bowtie, BWA, CLC bio, CloudBurst, Eland/Eland2, GenomeMapper, GnuMap, Karma, MAQ, MOM, Mosaik, MrFAST/MrsFAST, NovoAlign, PASS, PerM, RazerS, RMAP, SSAHA2, Segemehl, SeqMap, SHRiMP, Slider/SliderII, SOAP/SOAP2, Srprism, Stampy, vmatch, and ZOOM, etc. These methods may differ greatly in performance, such as aligning speed, memory requirements, and overall accuracy, and BWA is designed to achieve a good balance between performance and accuracy.

The BWA aligning algorithm is based on FM-index (Burrows-Wheeler Transform plus auxiliary data structures), which enables fast exact sequence matching. Its short-read algorithm is designed to alter the read sequence such that it matches the reference exactly. Its long-read algorithm (BWA-SW) takes sample reference subsequences and perform Smith-Waterman alignment between the subsequences and the read. BWA works for Illumina and SOLiD single-end (SE) and paired-end (PE) reads; BWA-SW works for 454/Sanger SE reads.

As a result, BWA is fast yet requires only moderate memory footprint (generally less than 4 GB); uses SAM output by default; has gapped alignment for both SE and PE reads; achieves high alignment accuracy using effective pairing (suboptimal hits are also considered in pairing). It treats non-unique read by placing it randomly with a mapping quality of 0, and all hits can be outputted in a concise format. Although most short reads (even 30 nucleotides in length) can be uniquely placed (see Rozowsky et al., Biotechnol., 27:66-75, 2009) onto the human genome, read placement may be challenging for reads that originate from repetitive regions or regions of segmental duplication. These reads can be aligned to multiple locations in the genome with equal (or almost equal) scores. Instead of simply excluding such unmappable genomic regions from consideration, BWA places such a read to a random location out of many where a read aligns with similar scores—a mapping quality of 0.

BWA is also guaranteed to find k-difference in the seed region (first 32 bp by default). The default configuration of BWA works for most typical sequence input. In addition, it automatically adjusts parameters based on read lengths and error rates, and estimates the insert size distribution on the fly.

The running of the BWA aligner can be briefly summarized below. First, an input with the format of ref.fa, read1.fq.gz, read2.fq.gz, or long-read.fq.gz is fed to the program. Then in Step 1: the reference genome is indexed (e.g., it takes about 3 CPU hours to index the human genome). Step 2a then generates alignments in the suffix array coordinate. If the quality is poor at the 3′-end of the reads, option “-q15” may be applied for improvement. Step 3a then generates alignments in the SAM format. Finally, Step 4a gets multiple hits. Alternatively, Step 2b uses BWA-SW for long reads.

The output of the BWA mapping file is the commonly known bam file, which can be used with the other sequencing analysis software described below to identify the various genomic variations.

Once the bam files for the SG fragment sequences and the MP fragment sequences are generated separately, the method of the invention utilizes these bam files (e.g., SG bam file and MP bam file) in conjunction with the various software packages to identify genetic variations.

For example, one software package that can be used in the method of the invention to preferentially identify small genetic variations, such as SNPs and indels, is the publically available “Genome Analysis Tool Kit” (or GATK) package developed by the Broad Institute. See McKenna et al., “The Genome Analysis Toolkit: a MapReduce framework for analyzing next-generation DNA sequencing data,” Genome Res., 20:1297-1303, 2010; DePristo et al., “A framework for variation discovery and genotyping using next-generation DNA sequencing data,”

Nat. Gen., 43:491-498, 2011; and Van der Auwera et al., “From FastQ Data to High-Confidence Variant Calls: The Genome Analysis Toolkit Best Practices Pipeline,” Curr. Prot. Bioinfo., 43:11.10.1-11.10.33, 2013 (all incorporated herein by reference).

GATK offers a wide variety of tools useful for analyzing high-throughput sequencing data. Taking advantage of the common architecture and powerful engine, the tools can be chained into scripted workflows to perform simple to complex “reads-to-results” analyses.

The primary focus of GATK is on variant discovery and genotyping, with a strong emphasis on data quality assurance. Since 2010, more than 150 research papers published in high impact scientific journals have successfully utilized GATK to solve various research questions. GATK has become an industrial standard for identifying mutations specific for a subpopulation. The software package can use data generated with a variety of different sequencing technologies, including the bam files of BWA for reads, quality scores, alignments, and metadata (e.g., the lane of sequencing, center of origin, sample name, etc.). GATK can also handle genome data from any organism (including human), and with any level of ploidy (such as plant genome with multiploidy).

In one embodiment, the method of the invention uses one of the variant discovery tools of GATK—the HaplotypeCaller—to identify SNPs and indels of an input bam file, such as the SG fragment bam file or the MP fragment bam file. In one embodiment, the input bam file is SG fragment bam file having at least 20-30 fold of sequence coverage, e.g., at least about 20-fold, 25-fold, 30-fold, 35-fold, 40-fold, 45-fold, or about 50-fold coverage. In certain embodiments, only SG bam file is used to identity SNPs and indels. In certain embodiments, only MP bam file is used to identify SNPs and indels. In certain embodiments, both SG and MP bam files are used to identify SNPs and indels.

The HaplotypeCaller tool calls SNPs and indels simultaneously via local re-assembly of haplotypes in an active region. It utilizes input bam file(s) from which to make calls, and produces an output VCF file with raw, unfiltered SNP and indel calls. These can then be filtered either by variant recalibration (best) or hard-filtering before use in downstream analyses. The basic operation of the HaplotypeCaller proceeds as follows:

1. Define Active Regions

The program determines which regions of the genome it needs to operate on, based on the presence of significant evidence for variation.

2. Determine Haplotypes by Re-Assembly of the Active Region

For each ActiveRegion, the program builds a De Bruijn-like graph to reassemble the ActiveRegion, and identifies what are the possible haplotypes present in the data. The program then realigns each haplotype against the reference haplotype using the Smith-Waterman algorithm in order to identify potentially variant sites.

3. Determine Likelihoods of the Haplotypes Given the Read Data

For each ActiveRegion, the program performs a pairwise alignment of each read against each haplotype using the PairHMM algorithm. This produces a matrix of likelihoods of haplotypes given the read data. These likelihoods are then marginalized to obtain the likelihoods of alleles for each potentially variant site given the read data.

4. Assign Sample Genotypes

For each potentially variant site, the program applies Bayes' rule, using the likelihoods of alleles given the read data to calculate the likelihoods of each genotype per sample given the read data observed for that sample. The most likely genotype is then assigned to the sample.

In a related embodiment, the method of the invention uses another variant discovery tools of GATK—the UnifiedGenotyper—to identify SNPs and indels of an input bam file, such as the SG fragment bam file or the MP fragment bam file. In one embodiment, the input bam file is SG fragment bam file having at least 20-30 fold of sequence coverage, e.g., at least about 20-fold, 25-fold, 30-fold, 35-fold, 40-fold, 45-fold, or about 50-fold coverage. In certain embodiments, only SG bam file is used to identity SNPs and indels. In certain embodiments, only MP bam file is used to identify SNPs and indels. In certain embodiments, both SG and MP bam files are used to identify SNPs and indels.

The UnifiedGenotyper is a variant caller which unifies the approaches of several disparate callers, and it works for single-sample and multi-sample data. The data input can be, among others, the bam file. The output is a raw, unfiltered, highly sensitive callset in VCF format. In certain embodiments, post-calling filters (such as Variant Quality Score Recalibration) are used to eliminate certain false positive calls. In certain embodiments, the generalized ploidy model is used to handle non-diploid or pooled samples.

In certain embodiments, the UnifiedGenotyper is used to identify SNP. In certain embodiments, the HaplotypeCaller is used to identify indels.

Compared to smaller genomic variations such as SNPs, accurate detection, genotyping and understanding of SVs/CNVs is lagging behind due to much greater analytical challenges related to SV/CNV detection and analysis. SVs and CNVs can be analyzed and detected using high-throughput sequencing data and different analytical approaches, such as those developed at the Yale University. For example, vcf2diploid is a personal genome constructor that can be used to construct a personal diploid genome sequence by including personal variants into a reference genome. See Rozowsky et al., “AlleleSeq: analysis of allele-specific expression and binding in a network framework,” Mol. Syst. Biol., 7:522. doi: 10.1038/msb.2011.54 (2011, incorporated by reference). CNVnator is a tool for CNV discovery and genotyping from depth of read mapping. See Mills et al., “Mapping copy number variation by population-scale genome sequencing,” Nature, 470(7332):59-65. doi: 10.1038/nature09708 (2011); and Abyzov et al., “CNVnator: an approach to discover, genotype, and characterize typical and atypical CNVs from family and population genome sequencing,” Genome Res., 21(6):974-84. doi: 10.1101/gr.114876.110 (2011) (both incorporated by reference). AGE is a tools that implements an algorithm for optimal alignment of sequences with SVs. See Abyzov and Gerstein, “AGE: defining breakpoints of genomic structural variants at single-nucleotide resolution, through optimal alignments with gap excision,” Bioinformatics, 27(5):595-603. doi:10.1093/bioinformatics/btq713 (2011) (incorporated by reference). BreakSeq is a pipeline for annotation, classification and analysis of SVs at single nucleotide resolution. See Lam et al, “Nucleotide-resolution analysis of structural variants using BreakSeq and a breakpoint library,” Nat. Biotechnol., 28(1):47-55. doi: 10.1038/nbt.1600 (2010) (incorporated by reference). PEMer is a computational and simulation framework for discovering SVs by paired-end read mapping. See Korbel et al., “PEMer: a computational framework with simulation-based error models for inferring genomic structural variants from massive paired-end sequencing data,” Genome Biol., 10(2):R23. doi: 10.1186/gb-2009-10-2-r23 (2009); and Korbel et al., “Paired-end mapping reveals extensive structural variation in the human genome,” Science, 318(5849):420-6 (2007) (both incorporated by reference).

In certain embodiments, CNVs are identified using the SG and/or the MP bam files using the publically available CNVnator package (freely available at http column double slash sv dot gersteinlab dot org slash cnvnator slash, and can be applied to various human and non-human genomes), which detects CNVs from a statistical analysis of mapping density, i.e., read-depth analysis (RD), of short reads from next-generation sequencing platforms. In contrast to previous RD-based approaches, which are limited to only unique regions of the genome for discovery of only large CNVs with poor breakpoint resolution, CNVnator is able to discover CNVs in a vast range of sizes, from a few hundred bases to megabases in length, in the whole genome. More specifically, for the calculation of the RD signal, CNVnator divides the whole genome into nonoverlapping bins of equal size, and uses the count of mapped reads within each bin as the RD signal. It then partitions the generated signal into segments with presumably different underlying copy numbers. Putative CNVs are predicted by applying statistical significance tests to the segments. The partitioning is based on a mean-shift technique originally developed in computer science for image processing.

Specifically, sequencing data of the SG and/or MP fragments can be obtained using any suitable sequencing methods, such as any of the NGS, including but not limited to Illumina/Solexa, Roche/454, and Life Technologies/SOLiD sequencing technology platforms. Such sequencing data is then used to generate SG/MP barn files. The CNVnator software package is then used to call/identify CNVs based on the SG barn file, the MP barn file, or both.

The SVs, including copy number neutral (non-CNV) SVs, can be identified using the methods of the invention by calling for such genomic variations using the SG and/or MP barn files using a method substantially identical to that described in Yao et al., “Long Span DNA Paired-End-Tag (DNA-PET) Sequencing Strategy for the Interrogation of Genomic Structural Mutations and Fusion-Point-Guided Reconstruction of Amplicons,” PLOS One, 7(9):e46152 (2012) (incorporated by reference). This method can identify SVs with a small insert size library (e.g., sub-kilobase range) associated with tight size selection of DNA fragments and greater sensitivity for small intra-chromosome rearrangements. The method can also identify larger insert size libraries (e.g., kilobase to tens of kilobases in range) associated with higher physical coverage of the genome, with the possible drawback of less precise localization of the breakpoint regions. That is, larger insert sizes have higher physical coverage and allow spanning across repetitive regions, thus tending to maximize the clonal coverage and detect as many rearrangement breakpoints as possible while reducing the sequence effort. On the other hand, smaller insert size provides better localization information, is advantageous in identifying deletions with span of less than 5 kb, and tends to identify larger number of deletions due to the more precise insert size selection and thereby smaller standard deviation of the insert size distribution. Furthermore, when used together as a combined library of several insert sizes, the probability of detecting a breakpoint with the combined library is higher than using only one type of insert size in the library.

Although large and small insert size libraries have comparable precision in locating breakpoints, large insert sizes also enabled better identification of SVs within repetitive sequences based on a fusion-point-guided-concatenation algorithm.

Thus in one embodiment, size selection can be used to construct circular genomic fragments of relatively smaller sizes (e.g., 1, 2, 3, 4, 5 kb, etc.). In other embodiments, size selection can be used to construct circular genomic fragments of relatively larger sizes (e.g., 5, 6, 7, 8, 9, 10, 15, 20, 25, 30, 35, 40, 45, 50 or more kb, etc.). In certain embodiments, circular genomic fragments of different/multiple size ranges are used in the methods of the invention.

Using the methods described above, sequencing data for the SV and MP fragments are compiled in the SG and MP bam files, for use in the SV detection methods described below.

In certain embodiments, the MP bam file is used in the method of the invention to detect SVs. The genomic DNA sequences flanking the tag sequences are also referred to as PETs (paired-end tags). Based on the mapping pattern of the sequence reads, the PETs can be distinguished as concordant PETs (cPETs) and discordant PETs (dPETs). The cPETs are defined as those PETs where both tags mapped to the same chromosome, the same strand, in the correct 5′ to 3′ ordering, and within expected span range (e.g., 3 kb for 1 kb library, 20 kb for 10 kb library, and 40 kb for 20 kb library, etc.). The PETs which were rejected by cPET criteria are classified as dPETs. Chimeric dPETs may be generated due to ligation error in the library construction process. To filter these out, dPET which span the same fusion point are required to form clusters. The number of the dPETs clustering together around a fusion point is represented by the cluster size or cluster count. The genomic region which is covered by the 5′ tags of a cluster is defined as the 5′ anchor and the genomic region which is covered by the 3′ tags of a cluster is defined as the 3′ anchor.

To identify SVs, SVs with one rearrangement point could be identified by single dPET clusters, such as deletions if the 5′ mapping anchor region is far away from the 3′ mapping anchor region, tandem duplications if the mapping order is 3′ to 5′ instead of the normal 5′ to 3′, unpaired inversion if the mapping orientation is revered (on different strand), and isolated translocations if the 5′ and 3′ anchors map to different chromosomes. Inversions, insertions and balanced translocations are identified by two closely positioned dPET clusters.

To separate breakpoints in complex regions from isolated and less complex SVs, a breakpoint based interconnection network can be established. The extension from the start and end points of each dPET cluster anchor region by the maximum insert size of the library is created as search windows to determine the neighborhood of a breakpoint. The dPET clusters are grouped as a supercluster when windows of neighboring clusters overlapped with each other. The number of dPET clusters that could be joined together into a supercluster is represented by supercluster size or supercluster count.

In certain embodiments, different size-selected insert sizes are used. In these embodiments, dPET clusters across different insert size libraries can be performed based on an overlap of the 5′ and 3′ anchor region extended by the individual library insert size. For example, to compare dPETs clusters across 10 kb and 20 kb insert size libraries, the 5′ and 3′ anchor regions of the cluster is extended by the maximum length of the library towards the breakpoints to create a search window. If the 5′ and 3′ anchor regions of a dPET cluster from other insert size libraries which belongs to the same SV type falls into the search window, the clusters would be grouped as a common SV. If no other cluster could be found in the search window, the cluster would be categorized as a SV specific to that insert size library.

In certain embodiments, the method of the invention further comprises using fluorescence in situ hybridization (FISH) to verify the identified SVs, or to place the SVs in a cytogenetic context.

In certain embodiments, the methods of the invention further comprises verifying breakpoints of the identified SVs by, e.g., genomic PCR and Sanger sequencing.

In certain embodiments, the method of the invention further comprises reconstructing the whole genome rearrangement or the identified SVs by using fusion-point-guided-concatenation algorithm. In particular, segmenting of the reference genome into contigs is assembled based on the breakpoints identified by dPET clusters and by identifying additional breakpoints with no physical cPET coverage. Contigs consecutive on the reference genome are then connected by a reference edge in the presence of connecting cPETs. Correspondingly, contigs linked by dPET clusters are represented by dPET edges where the edges are weighted by the size of the cluster. Locally amplified regions are then identified in the following way: Firstly, the dPET edge with the highest weight is selected and the adjacent contigs to this edge are added to the amplicon graph. Then, for each contig in the graph, its neighbors are also added using both reference and dPET links as long as the neighbors are considered amplified (cPET estimated copy-number greater than 2). An amplicon graph is grown until no more contigs could be added in this fashion. The process is then repeated on the unused dPET edges, till none remained, resulting in a set of local amplicon graphs and only graphs with more than two contigs are considered further.

### 4. Detection of Genomic Variations in Diseases and Disorders

The methods of the invention can be used to detect all types of genomic variations in a single assay from any organism. The methods of the invention are particularly useful in identifying such genomic variations in certain human diseases or disorders known to have complicated underlying genomic defects.

In certain embodiments, the methods of the invention can be used to detect genomic variations in Autism Spectrum Disorder (ASD) patients, or patients suspected of having ASD or at high risk of developing ASD.

ASDs are increasingly being diagnosed as a collection of linked developmental disorders, characterized by abnormalities in social interaction and communication, restricted interests, and repetitive behaviors. In addition to classical autism or Autistic Disorder, the fifth edition of the American Psychiatric Association's (APA) Diagnostic and Statistical Manual of Mental Disorders (DSM-5) recognizes Asperger syndrome, Childhood Disintegrative Disorder, and Pervasive Developmental Disorder Not Otherwise Specified (PDD-NOS) as ASDs.

Like schizophrenia, mutations in over 100 different loci have been found in ASD, making the methods of the invention particularly suitable to unravel the complicated underlying genetic defects in any individual patient of ASD.

ASD is one type of neurodevelopmental disorders (NDDs), the latter of which also include Fragile X Syndrome (FXS), Angelman Syndrome, Tuberous Sclerosis Complex, Phelan McDermid Syndrome, Rett Syndrome, CDKLS mutations (which also are associated with Rett Syndrome and X-Linked Infantile Spasm Disorder) and others. Many but not all NDDs are caused by genetic mutations. Some patients with NDDs exhibit behaviors and symptoms of autism. Thus the methods of the invention may also be used in these NDDs.

In certain embodiments, the methods of the invention can be used to detect genomic variations in other complex diseases that result from interactions between multiple genes, or genes and the environment. Such complex diseases may include, without limitation, Alzheimer's disease, asthma, Parkinson's disease, diabetes, obesity, heart conditions, cancers, high blood pressure, other familiar diseases of the heart and circulatory system, psychiatric illness such as schizophrenia and depression, inflammatory autoimmune diseases such as arthritis and Crohn's disease, multiple sclerosis, and others.

### EXAMPLES

### Example 1

Using the methods of the invention, various genomic variations in an autism patient P46107 were identified, and the characterized genomic variations are tabulated based on size in the table below. “DNA-PET” stands for MP sequencing data.

Specifically, the patient sample was obtained from a hospital, and the sample was anonymized prior to sequencing and analysis. Genomic DNA was extracted from the sample using AllPrep DNA/RNA Mini Kit (Qiagen) according to the manufacturer's instruction. The DNA sequencing library was prepared using the methods of the invention as described above. Briefly, the genomic DNA sample was simultaneously fragmented and tagged with junction adaptor using Illumina formulated mate pair transposome. After the tagmentation, a polymerase was used to fill in the short single stranded sequence gap in the tagmented DNA by strand displacement reaction. Genomic DNA fragments of between 6 to 8 kb were selected by Sage Pippin Prep. The size-selected fragments were then circularized in a blunt ended intramolecular ligation, with an overnight incubation optimized to maximize the number of fragments that will form circular molecules. The circularized DNA fragments were then physically sheared to approximately 400-500 bp average size fragments. End repair and A-tailing reactions were performed on the sheared fragments, before the Illumina TruSeq adaptors were ligated to the fragmented DNAs. The fragmented DNAs were sequenced by 2×150 bp by Illumina Hi-Seq 2500 according to the manufactory's recommendations.

Using the junction adaptor in the sequence, the MP and SG fragment sequences were sorted out separately based on sequence analysis. The MP and SG sequences were then mapped to the reference human genome, respectively, to generate two bam files. The mapped SG and/or MP bam files were then used for all genetic variation detections as described above. The detected genomic variations from the sample are categorized and summarized in the table below.

| | Detect by DNA- | | | | | |
|---|---|---|---|---|---|---|
| Del | PET | | Detect by SG | | Detect by both | |
| Size | Number | Ratio (%) | Number | Ratio (%) | Number | Ratio (%) |
| <1 kb | 0 | 0 | 1782 | 65.9 | 0 | 0 |
| 1-5 kb | 0 | 0 | 614 | 22.7 | 0 | 0 |
| 5-10 kb | 61 | 31.8 | 140 | 5.2 | 44 | 42.7 |
| 10-20 kb | 96 | 50 | 42 | 1.6 | 37 | 35.9 |
| 20-100 kb | 28 | 14.6 | 64 | 2.4 | 21 | 20.4 |
| >100 kb | 7 | 3.6 | 64 | 2.4 | 1 | 1.0 |
| Total | 192 | 100 | 2706 | 100 | 103 | 100 |

It is apparent that the MP sequencing data is best suited for detecting larger size deletions (e.g., 5 kb and above), while the SG sequencing data is more appropriate for identifying smaller sized deletions (5 kb or less). Some variations can also be detected by both SG and MP sequencing data. This suggests that all types of genomic variations, both large and small in scale, can be efficiently detected by the method of the invention using a single sequencing run from one patient sample.

### Example 2

Using the methods of the invention, various genomic variations in five autism patients were identified, and the results were compared to those identified from the same patients using the current standard assays based on array CGH and exon sequencing.

The comparison showed that, for each CNV structure variation identified by the traditional aCGH assay, there is a perfect match identified by the methods of the invention. However, the methods of the invention identified much more genomic variations not identified by aCGH, thus representing an opportunity for identifying more new variants using the methods of the invention.

For example, for Patient DBS0005 (Autism Spectrum Disorder), a Transgenomic® Postnatal High Density SNP Array Test revealed that there is a 383.4 kb deletion in the chromosomal region of 5q23.3, including genes LYRM7 and HINT1. Using the methods of the invention, a 383.591 bp deletion in the same chromosomal region (Chr5: 130140673-130520365) was identified.

In another example, for Patient DBS0010 (Autism, with speech delay), a GeneDX GenomeDx Report of whole genome array CGH+SNP analysis revealed that the patient carries a duplication of at least 302 kb of a region within cytogenetic band 12q24.33, which duplicated interval contains 7 known genes. Using the method of the invention, a 312,717 bp tandem duplication in the same chr.12 region (133091631-133393167) was identified.

The method of the invention also identified the following patient specific deletions not identified by traditional methods aCGH. Part of the reason for the methods of the invention to be able to identify much more genomic variations is because aCGH has significant resolution limitations, such that it can only reliably detect deletions larger than 200 kb, while the methods of the invention can detect deletions with much higher resolution, from a few hundreds base-pairs to up to hundreds of kbs.

| #chrom | start | end | PET | lib data | length | patients |
|---|---|---|---|---|---|---|
| chr5 | 130135661 | 130519252 | 3 | 05MP | 383591 | 1 |
| chr19 | 22247416 | 22354747 | 5 | 05MP | 107331 | 1 |
| chr6 | 32627700 | 32728875 | 3 | 11MP | 101175 | 1 |
| chr3 | 46792449 | 46855433 | 2 | 10MP | 62984 | 1 |
| chr14 | 41608541 | 41670629 | 5 | 07MP | 62088 | 1 |
| chr5 | 180372247 | 180432857 | 5 | 07MP | 60610 | 1 |
| chr18 | 65845338 | 65898923 | 5 | 11MP | 53585 | 1 |
| chr17 | 36350127 | 36401848 | 4 | 08MP | 51721 | 1 |
| chr13 | 57748565 | 57793423 | 13 | 10MP\|05MP\|11MP\|08MP | 43354 | 4 |
| chr3 | 165260606 | 165301500 | 4 | 10MP | 40476 | 1 |
| chr14 | 106881396 | 106921067 | 11 | 10MP\|11MP\|08MP | 39671 | 3 |
| chr9 | 26273861 | 26307251 | 3 | 08MP | 33390 | 1 |
| chr11 | 5781499 | 5809819 | 9 | 08MP\|07MP | 28320 | 2 |
| chr11 | 7808451 | 7836017 | 3 | 05MP | 27566 | 1 |
| chr7 | 98327136 | 98354556 | 5 | 07MP | 27420 | 1 |
| chr8 | 75306918 | 75332958 | 2 | 10MP | 26040 | 1 |
| chr6 | 77436241 | 77462270 | 10 | 07MP\|11MP | 25830 | 2 |
| chr4 | 64691440 | 64715803 | 5 | 07MP | 24363 | 1 |
| chr7 | 120711692 | 120737159 | 4 | 10MP | 23690 | 1 |
| chr9 | 5384921 | 5408601 | 4 | 05MP | 23680 | 1 |
| chr8 | 2246798 | 2270107 | 5 | 10MP | 23309 | 1 |

* Patients 1-5 are DBS0005, 0007, 0008, 0010, and 0011, respectively.
There are altogether 273 deletions of >10 kb; and 29 deletions of >20 kb.

Similarly, for SNPs, of the 51 reported by the traditional exon sequencing, 49 were also identified by the methods of the invention—a 96% match. In fact, for the 2 SNP differences, it is not certain if they are due to false positive identification by the exon sequencing method, or due to false negative identification by the methods of the invention.

Specifically, Courtagen gene panel SNP data was compared to the SNPs identified by the methods of the invention, and the results in the 5 patients are summarized below.

| | Courtagen | Applicant | Match (%) |
|---|---|---|---|
| DBS0005 | 7 | 7 | 100 |
| DBS0007 | 6 | 6 | 100 |
| DBS0008 | 3 | 3 | 100 |
| DBS0010 | 4 | 3 | 75 |
| DBS0011 | 4 | 3 | 75 |

More specifically, in patient DBS0005, the following SNPs in the following genes were identified by Courtagen and the methods of the invention:

| Gene | Courtagen | Applicant | Match |
|---|---|---|---|
| CREBBP | G/A | G/A | Yes |
| HOXA1 | T/C | T/C | Yes |
| MAP2K2 | G/A | G/A | Yes |
| MET | T/C | T/C | Yes |
| NHS | C/T | C/T | Yes |
| RELN | C/T | C/T | Yes |
| TSC1 | G/A | G/A | Yes |

In patient DBS0007, the following SNPs in the following genes were identified by Courtagen and the methods of the invention:

| Gene | Courtagen | Applicant | Match |
|---|---|---|---|
| KIAA2022 | G/A | G/A | Yes |
| MBD5 | G/A | G/A | Yes |
| MED12 | C/T | C/T | Yes |
| MKKS | C/T | C/T | Yes |
| NIPBL | G/A | G/A | Yes |
| VPS13B | C/T | C/T | Yes |

In patient DBS0008, the following SNPs in the following genes were identified by Courtagen and the methods of the invention:

| Gene | Courtagen | Applicant | Match |
|---|---|---|---|
| MED12 | G/A | G/A | Yes |
| MED23 | TTC/T | TTC/T | Yes |
| RAF1 | C/T | C/T | Yes |

In patient DBS0010, the following SNPs in the following genes were identified by Courtagen and the methods of the invention:

| Gene | Courtagen | Applicant | Match |
|---|---|---|---|
| NRXN1 | G/A | G/A | Yes |
| SGSH | G/C | G/C | Yes |
| TRAPPC9 | C/T | C/T | Yes |
| TSC2 | T/C | NONE | NO |

In patient DBS0011, the following SNPs in the following genes were identified by Courtagen and the methods of the invention:

| Gene | Courtagen | Applicant | Match |
|---|---|---|---|
| GRIN2B | G/C | G/C | Yes |
| NAGLU | C/T | C/T | Yes |
| SCN1A | C/T | NONE | Yes |
| TSC2 | A/G | A/G | NO |

In short, based on these 5 patient datasets, the methods of the invention worked extremely well, and demonstrated great potential to replace the multiple existing standard assays as the new standard for identifying all genomic variations.

1. A method for detecting genomic variations in the genome of an organism, the method comprising:
(1) fragmenting genomic DNA of the organism to generate a plurality of genomic DNA fragments;
(2) tagging the ends of the genomic DNA fragments with a tag sequence;
(3) ligating tagged ends of the genomic DNA fragments, under a condition that promotes blunt-end intramolecular ligation, to generate a plurality of circularized genomic DNA fragments with ligated tag sequence;
(4) fragmenting the plurality of circularized genomic DNA fragments by shotgun fragmentation, to generate:
(a) a plurality of mate-pair (MP) fragments, each comprising the ligated tag sequence flanked by flanking genomic DNA; and,
(b) a plurality of shotgun (SG) fragments;
(5) determining the sequences of the MP fragments and the SG fragments; and,
(6) identifying said genomic variations in the genome of the organism based on both the sequences of the SG fragments and the sequences of the MP fragments.

2. The method of claim 1, wherein said genomic variations comprise one or more of: single nucleotide polymorphisms (SNPs); small insertions or deletions (indels); tandem base mutations (TBM); copy number variations (CNVs); structural variations (SVs); and combination thereof.

3. The method of claim 1, wherein steps (1) and (2) are carried out simultaneously.

4. The method of claim 3, wherein steps (1) and (2) are effected by transposon-mediated tagmentation.

5. The method of claim 4, wherein transposon-mediated tagmentation is carried out by a Tn5 transposase.

6. The method of claim 1, wherein the plurality of genomic DNA fragments is size-selected prior to step (3).

7. The method of claim 6, wherein genomic DNA fragments of about 4-10 kb, or about 6-8 kb, are size-selected.

8. The method of claim 1, wherein uncircularized or linear genomic DNA fragments are removed by DNA exonuclease digestion prior to steps (4)-(6).

9. The method of claim 1, wherein sequences of the MP fragments and the SG fragments are determined separately or simultaneously.

10. The method of claim 1, wherein the SG fragments have an average size of about 400 bp, 450 bp, or 500 bp.

11. The method of claim 1, wherein the MP fragments have an average size of about 400 bp, 450 bp, or 500 bp.

12. The method of claim 1, wherein the MP fragments and the SG fragments are isolated from each other before step (5).

13. The method of claim 1, wherein the MP fragments and the SG fragments are not isolated from each other before step (5).

14. The method of claim 1, wherein tagged ends of the genomic DNA fragments are repaired to promote blunt end ligation prior to step (3).

15. The method of claim 1, wherein step (6) comprises mapping the sequences of the flanking genomic DNA and the sequences of the shotgun fragments to the genomic sequence of the organism.

16. The method of claim 1, wherein sequences of the genomic DNA is determined by high-throughput sequencing.

17. The method of claim 16, wherein the high-throughput sequencing is selected from the group consisting of: single-molecule real-time sequencing; ion semiconductor (Ion Torrent) sequencing; pyrosequencing (454); sequencing by synthesis (Illumina); sequencing by ligation (SOLiD sequencing); polony sequencing; massively parallel signature sequencing (MPSS); DNA nanoball sequencing; single molecule nanopore sequencer; and Heliscope single molecule sequencing.

18. The method of claim 16, wherein the high-throughput sequencing produces 30-, 40-, 50-, 60-, 70-, 80-, 90-, 100- or more fold of coverage for the flanking genomic DNA and/or the shotgun fragments.

19. The method of claim 1, wherein the organism is a human, a non-human primate, a mammal, a rodent (rat, mouse, hamster, rabbit), livestock animal (cattle, pig, horse, sheep, goat), a bird (chicken), a reptile, an amphibians (Xenopus), a fish (zebrafish (Danio rerio), puffer fish), an insect (Drosophila, mosquito), a nematode, a parasite, a fungus (yeast, such as S. cerevisae or S. pombe), a plant, a bacterium, or a virus.

20. The method of claim 1, wherein the organism is a human having a disease or condition selected from the group consisting of: autism (autism spectrum disorder (ASD)), cancer, or hereditary disease.
